(19) United States
(12) Patent Application Publication
(10) Pub. No.: US 2018/0346513 A1
(43) Pub. Date: Dec. 6, 2018

(54) METHOD FOR OBTAINING TUMOR PEPTIDES AND USES THEREOF

(71) Applicant: ISTITUTO EUROPEO DI ONCOLOGIA S.R.L., Milano (MI) (IT)

(72) Inventors: Giuseppe PENNA, Milano (MI) (IT); Maria RESCIGNO, Milano (MI) (IT)

(21) Appl. No.: 15/775,592

(22) PCT Filed: Nov. 11, 2016
(86) PCT No.: PCT/EP2016/077483

§ 371 Date: May 11, 2018

(30) Foreign Application Priority Data
Nov. 11, 2015 (EP) EP15194078.0

(51) Int. Cl. C07K 7/06; C12N 5/0783; C12N 5/0784; C12N 5/09; C07K 7/08

(52) U.S. Cl. CPC C07K 7/06; C12N 5/0638; C12N 5/0639; C12N 5/0693; A61K 39/00; C12N 2502/1121; C12N 2502/1114; C12N 2501/24; C07K 7/08

(57) ABSTRACT

The present invention refers to a method for obtaining a cell culture supernatant or a fraction thereof having a specific tumor antigen peptide repertoire having the steps of: a) exposing in suitable conditions a tumor cell culture expressing the specific tumor antigen peptide repertoire to a Pattern Recognition Receptor (PRR) agonist and/or to one inflammatory cytokine to increase the opening of connexin-hemichannels (CxH); b) collecting the cell culture supernatant; and c) optionally obtaining a fraction of the supernatant, wherein the supernatant has MHC class I and MHC class II peptides and non-classical MHC molecules (HLA-E).

The invention relates to a method of cancer vaccine generation. The invention relates to a methodology to open up connexin-hemichannels (CxH) in cancer cells. These channels can be successfully exploited to release tumor antigenic peptides directly in the extracellular milieu. Tumor peptides are able to induce an antitumor immune response in vivo, include both B and T cell novel epitopes. If injected in vivo such peptides foster both antibody and cellular responses against tumor cells.

### BACKGROUND

Peptide vaccines are preparations made from synthetic epitopes that represent the minimal immunogenic region of a protein. This therapy is mainly applied to melanoma since it is one of the most immunogenic cancers.

The major advantages of peptide vaccines are that they are simple, safe, stable and economical, as well as easy to produce and store. They are easily characterized and analyzed by well-established techniques (Mocellin 2012). Also, peptides can be relatively easily modified in order to improve their immunogenicity, stability and solubility by the introduction of lipid, carbohydrate and phosphate groups. The major weakness of peptide-based vaccines is their inconsistent ability to stimulate effector T cells: although they are quite able to stimulate TA-specific CD4+ T cells, they are only poorly able to stimulate TA-specific CD8+ T cells. The use of naturally occurring peptides alone for anticancer vaccination is rarely followed by a tumor response, so various approaches were studying to overcome this limitation (Adams, Lowes et al. 2008). One approach can be the use of immunological adjuvants: the slow release of antigens have been recognized as a critical method in the induction of effective immune responses. Among the adjuvants in current use with cancer vaccines are aluminium salt, oil in water emulsion and nontoxic derivates from Salmonella and the saponins. A major new advance in this field has been the introduction of toll-like receptor ligands (TLRL) which potently activate APCs in vivo. There are various toll-like receptor agonists currently in use such as TLR7/8L (Imiquimod, Resiquimod) and TLR9L (CpG). Notably, some TLRLs such as TLR3L have pleiotropic effects, activating APCs as well as NK, and mediating tumor cell death (Fourcade, Sun et al. 2010). CpG is a potent adjuvant for peptide cancer vaccines, stimulating ex vivo detectable TA-specific CD8+ T cells in patients with advanced cancer (Speiser, Baumgaertner et al. 2008).

Another approach to improve the immunogenicity of peptides is to alter amino acids at HLA binding residues (called “anchor residues”) to enhance their HLA binding affinity. These modifications can dramatically influence the conformation of the peptide/HLA groove inducing stronger anti-tumor immunity in mice and enhances the efficacy of T cell-induction in humans (Meijer, Dols et al. 2007). However, several findings suggest caution in the use of modified TAA (Tumor Associated Antigen) epitopes because in some cases they induce response in vivo that reduce the ability of immune system to control tumor growth.

A further approach is based on use of mimotopes, molecules that mimic an antigenic determinant of the nominal antigen and are capable of inducing antibody and cellular immune responses to the nominal antigen (Knittelfelder, Riemer et al. 2009) The rational of this strategy depends on the fact that mimotopes provide an alternative to natural T-cell epitopes for anticancer vaccination because they can recruit and stimulate T cell-repertoires that deviate from the repertoires engaged at the tumor site. Besides demonstrating therapeutic efficacy in preclinical models including melanoma, this approach has entered the clinical phase of experimentation (van Stipdonk, Badia-Martinez et al. 2009).

Additionally, a strategy widely applied for melanoma therapy is multi-epitope vaccine, which is believed to circumvent some limits of single epitope regimens. From the antimelanoma efficacy viewpoint, multiepitope vaccines have been shown to elicit adequate immune responses ex vivo (Parmiani, Castelli et al. 2002). However, modest or not significant therapeutic benefit has been so far reported in patients with advanced melanoma. Therefore, more work is needed in order to identify the correct formulation that might lead to therapeutic efficacy of peptide vaccine strategy. As a mechanism of immune evasion, tumor cells often have impaired ability to process and present tumor antigenic peptides (Chen, H. L. et al. Nat. Genet. 13, 210-213 (1996). Evans, M. et al. J. Immunol. 167, 5420-5428 (2001). Alimonti, J. et al. Nat. Biotechnol. 18, 515-520 (2000)). Several steps in tumor antigen processing can be affected from their processing to their transport into the endoplasmic reticulum by TAP. This allows tumor cells to evade recognition by tumor-specific T cells. However, in these circumastances a new class of peptides called T-cell epitopes associated with impaired peptide processing, TEIPP, can be presented on the cell surface as there is lack of competition for presentation on MHC molecules (both classical and non-classical) by TAP-transported peptides. TEIPP have been proposed as a possible tool to induce an immune response to tumors (Van Hall T et al. NATURE MEDICINE VOLUME 12 [NUMBER 4 [APRIL 2006). However, there were little or no tools to study the identity of these peptides.

Dillman R. et al (Cancer Biotherapy & Radiopharmaceuticals, 2009, 24, p. 311) relates to an autologous tumor cell vaccines consisting of dendritic cells (DCS), derived from patient's peripheral blood cells cultured in (IL)-4 and granulocyte macrophage colony-stimulating factor (GM-CSF), which had phagocytosed irradiated autologous tumor cells from a continuously proliferating, self-renewing, autologous tumor cell (TC) culture.

WO2009/040413 relates to a process to obtain activated antigen-presenting cells that are useful for therapies against cancer and immune system-related diseases, by means of a cellular composition that contributes to stimulate the activated antigen-presenting cells to induce specific immune response against tumours. The method induces the differentiation of monocytes in APC (dendritic cells) by stimulation in culture using cytokines, growth factors and/or mixture of lysate or extracts of tumour cells.

Mendoza-Naranjo A, et al, and Salazar-Onfray, J. Immunol. 2007; 178; 6949-6957 describes the use of melanoma cell lysate stimulated with TNFalfa to induce gap junctions to promote Ag transfer between ex vivo produced hDCs from melanoma patients.

WO2008019366 relates to methods and compositions for increased priming of T-cells through cross presentation of exogenous antigens. It refers to particles (S. Cerevisiae) on the surface of which the antigen is attached, and administering the antigen preparation to the animal, wherein the particles are taken up by antigen presenting cells (APC) of the animal via phagocytosis.

US 20090324651 relates to methods for stimulating an immune response using bacterial antigen delivery system. It relates to the use of the type III secretion system of bacteria to stimulate immune responses against tumor antigen(s) for treating antigen-loss variant tumors. Methods are provided for stimulating and/or increasing an immune response against tumor antigens.

The prior art document also relates to the preparation of antigen presenting cells from peripheral blood mononuclear cells using bacteria having a type III secretion system. The method refers to the culture of PBMCs, previously contacted with an avirulent bacteria (such as S. typhimurium) expressing a tumor antigen, and isolating antigen presenting cells. Salmonella acts as vehicle of the tumoral antigen (previously “loaded” on bacteria) to the APC cell (degradation of antigen is still made by the APC).

Eugenin E. A. et al, and Juan C. Saez. J. Immunol 2003, 170:1320-1328 discloses that TNF-alfa plus IFN-gamma induce connexin 43 expression and formation of gap junctions between human monocytes and macrophages.

Elgueta R. et al, and Saez J. J Immunol 2009, 183(1):277-84 reports the formation of gap junctions between DCs and T cells and their role on T cell activation during Ag presentation by DCs.

WO2004/050855 relates to a one-step method for producing antigen loaded dendritic cells vaccine comprising an activator such as TNF alpha preferably in combination with at least one growth factor such as GM-CSF and at least one soluble or particulate antigen.

AU2014271235 relates to peptides, nucleic acids and cells for use in immunotherapeutic methods. In particular, the invention relates to the immunotherapy of cancer. The invention furthermore relates to tumor associated cytotoxic T cell (CTL) peptide epitopes, alone or in combination with other tumor-associated peptides that serve as active pharmaceutical ingredients of vaccine compositions that stimulate anti-tumor immune responses. The invention relates to 30 peptide sequences and their variants derived from HLA class I and class II molecules of human tumor cells that can be used in vaccine compositions for eliciting anti-tumor immune responses.

WO2007028573 relates to tumour-associated T-helper cell peptide epitopes, alone or in combination with other tumour-associated peptides, that serve as active pharmaceutical ingredients of vaccine compositions which stimulate anti-tumour immune responses. In particular, the application relates to two novel peptide sequences derived from HLA class II molecules of human tumour cell lines which can be used in vaccine compositions for eliciting anti-tumour immune responses.

WO98/15282 relates to an immunogenic composition comprising at least one protein from TLP (tumor-liberated particles, proteic complexes present in human tumor cells) or a fragment thereof, and in particular to the compositions wherein said fragments can comprise at least one of the peptides defined by particular sequences, suitable in therapy against tumoral diseases, and in particular against NSCLC and uro-genital cancer.

WO94/01458 relates to peptides comprised within the 100 KDa protein of the TLP complex (i.e., released proteins from tumors) having antigenic activity as well as antibodies thereof, able to react with TLP for diagnostic and clinical purposes.

WO2009102909 provides tumor-associated HLA-restricted antigens, and in particular HLA-A2 restricted antigens, as immunogenic compositions for treating and/or preventing breast cancer in an individual. In specific aspects, PR1 peptide or a derivative thereof, or a myeloperoxidase peptide, or a cyclin E1 or E2 peptide is provided in methods and compositions for breast cancer treatment and/or prevention. Such peptides can be used to elicit specific CTLs that preferentially attack breast cancer based on overexpression of the target protein cells.

WO 2012/017033 and Saccheri, Pozzi et al. 2010 refer to infection of melanoma cells with Salmonella typhimurium which induces the up-regulation of connexin 43 (Cx43). Said up-regulation is correlated with the generation of functional gap junctions between tumor cells and dendritic cells. Tumor cells, via gap junctions, transfer pre-processed antigenic peptides to the DCs which activate cytotoxic T cells specific for the tumor antigens inducing an antitumor response in vivo. WO 2012/017033 does not refer to cell culture supernatant or its collection in the described method. Moreover the Application doesn't disclose the use of a cell culture supernatant as antitumor vaccine.

Dendritic cells (DCs) are key players in the activation of T cells. DCs comprise a family of antigen presenting cells, including plasmacytoid and conventional (myeloid) DCs. DCs are endowed with the ability to present exogenous antigens that have not been generated within DCs for the activation of T cells, via the cross-presentation pathway. Cross-presentation is required for the initiation of effective anti-tumor T cell responses and the repertoire of presented peptides is crucial to activate T cells that will recognize and kill tumor cells. However, the antigen presentation machinery, and in particular the proteasome, differs between tumor cells and dendritic cells. A major drawback is that DCs could process and present peptides that are different from those presented by tumor cells, thus initiating a tumor-specific response that will not recognize the tumor.

Gap junctions (GJs) are channels that connect the cytoplasm of two adjacent cells (B. J. Nicholson, J Cell Sci 116, 4479 (Nov. 15, 2003)). They allow the transfer of small molecules including ions, second messengers and metabolites up to 1 kDa (B. J. Nicholson, J Cell Sci 116, 4479 (Nov. 15, 2003)). GJ intercellular communication (GJIC) has been shown to participate to many physiological events like cell cycle control, differentiation, cell synchronization and metabolic coordination (B. J. Nicholson, J Cell Sci 116, 4479 (Nov. 15, 2003), G. Mese, G. Richard, T. W. White, J Invest Dermatol 127, 2516 (November, 2007).). GJs are formed by two hemichannels, called connexons, each made of six Connexin proteins. There are at least 21 Connexins most of which are tissue specific except for Connexin (Cx) 43 that is ubiquitously expressed (J. Neijssen, B. Pang, J. Neefjes, Prog Biophys Mol Biol 94, 207 (May-June, 2007).). Loss of GJIC is a common feature in many human tumors and can occur early during tumorigenesis (T. J. King, J. S. Bertram, Biochim Biophys Acta 1719, 146 (Dec. 20, 2005)., M. Mesnil, S. Crespin, J. L. Avanzo, M. L. Zaidan-Dagli, Biochim Biophys Acta 1719, 125 (Dec. 20, 2005).). Recently, GJs have been shown to play a prominent role also in the immune system (J. Neijssen, B. Pang, J. Neefjes, Prog Biophys Mol Biol 94, 207 (May-June, 2007).). They are required for B and T cell differentiation, antibody secretion by B cells, T regulatory cell activity (T. Bopp et al., J Exp Med 204, 1303 (Jun. 11, 2007).) and dendritic cell activation (E. Oviedo-Orta, W. Howard Evans, Biochim Biophys Acta 1662, 102 (Mar. 23, 2004)., H. Matsue et al., J Immunol 176, 181 (Jan. 1, 2006).). GJs are also involved in antigen cross-presentation by allowing the spreading of small linear peptides (up to 16 amino acid long) between neighboring cells (J. Neijssen et al., Nature 434, 83 (Mar. 3, 2005).), including apoptotic cells (B. Pang et al., J Immunol 183, 1083 (Jul. 15, 2009).). In absence of cell-cell contact GJ channels exist in form of hemichannels at nonjunctional membranes. Connexin hemichannels (CxHcs) in the plasma membrane are in a closed conformation under resting conditions but can be opened under the influence of stimuli such as low extracellular Ca2+, membrane depolarization, mechanical membrane stress and metabolic inhibition (Quist, Rhee et al. 2000) (Stout, Costantin et al. 2002) (Parpura, Scemes et al. 2004) (Cherian, Siller-Jackson et al. 2005).

It is still felt the need of a method providing the release in the culture medium of tumor cells, of peptides processed by the tumor proteasome with no HLA restriction and no risk of contaminating cancer cells, which could be loaded directly on DCs from the outside of the cell on surface MHC molecules.

### DESCRIPTION OF THE INVENTION

Inventors have surprisingly found that infection of tumor cells with Salmonella typhimurium induces the opening of connexin hemichannel allowing the release of pre-processed antigen peptides in the supernatant. The supernatant can be collected and used as antitumor vaccine.

Inventors found that said supernatant includes poorly represented peptides including TEIPP (‘T-cell epitopes associated with impaired peptide processing’).

Therefore, inventors shown that by means of bacterial infection it is possible to open Cx43 hemichannels through which antigenic peptides can be released in the extracellular milieu. These peptides have been processed by the tumor proteasome and, unexpectedly, they include besides MHC class I peptides, also MHC class II peptides for both CD4 and CD8 activation as well as non-classical MHC molecules (HLA-E).

Human Leukocyte Antigen (HLA)-E is a low-polymorphic non-classical HLA class I molecule which plays a crucial role in immune surveillance by presentation of peptides to T and natural killer (NK) cells.

By including MHC class II peptides, the released peptides are able to induce an humoral response. In addition, these peptides can be loaded on DCs from the outside of the cell on surface MHC molecules. This allows to bypass the TAP transporter and consequently allows to present also peptides that would never be presented using the classical processing pathway such as TEIPP peptides.

Therefore, it is an object of the invention a method for obtaining a cell culture supernatant or a fraction thereof comprising a specific tumor antigen peptide repertoire comprising the steps of:

a) exposing in suitable conditions a tumor cell culture to at least one Pattern Recognition Receptor (PRR) agonist and/or to one inflammatory cytokine to increase the opening of connexin-hemichannels (CxH);
b) collecting the cell culture supernatant; and
c) optionally obtaining a fraction of said supernatant,
wherein said supernatant comprises MHC class I and MHC class II peptides and non-classical MHC molecules (HLA-E).

A further object of the invention is a method for obtaining a specific tumor antigen peptide repertoire loaded and/or activated dendritic cell comprising the steps of:

a) exposing in suitable conditions a tumor cell culture to at least one Pattern Recognition Receptor (PRR) agonist and/or to one inflammatory cytokine to increase the opening of connexin-hemichannels (CxH);
b) collecting the cell culture supernatant;
c) culturing dendritic cells with the collected cell culture supernatant, or a fraction thereof or with at least one purified peptides from said cell culture supernatant, to get specific tumor antigen peptide repertoire loaded and/or activated dendritic cells; and
c) optionally purifying said specific tumor antigen peptide repertoire loaded and/or activated dendritic cells.

Another object o the invention is a method for obtaining an activated tumor antigen-specific CTL comprising the steps of:

a) exposing in suitable conditions a tumor cell culture to at least one Pattern Recognition Receptor (PRR) agonist and/or to one inflammatory cytokine to increase the opening of connexin-hemichannels (CxH);
b) collecting the cell culture supernatant;
b) co-culturing dendritic cells and CTLs with the cell culture supernatant, or a fraction thereof or with at least one purified peptides from the cell culture supernatant, to get activated tumor antigen-specific CTLs.

In the method for obtaining activated tumor antigen-specific CTL, dendritic cells are preferably incubated with a sample of CD8+ T cells isolated from a donor.

Preferably, the dendritic cells are autologous or HLA-compatible or semi-compatible allogenic dendritic cells.

In the methods according to the invention, in step a) the tumor cell culture are preferably incubated for at least 30 minutes with at least one Pattern Recognition Receptor (PRR) agonist and/or to one inflammatory cytokine to increase the opening of connexin-hemichannels (CxH).

Preferably, the tumor cell culture is incubated at a temperature of 25-50° C. with at least one Pattern Recognition Receptor (PRR) agonist and/or to one inflammatory cytokine to increase the opening of connexin-hemichannels (CxH).

More preferably, in step a) of the above methods the tumor cell culture is incubated for 1 hour and half at 37° C. with at least one Pattern Recognition Receptor (PRR) agonist and/or to one inflammatory cytokine to increase the opening of connexin-hemichannels (CxH).

Said cell culture supernatant is preferably obtained by centrifugation of cells.

More preferably, after centrifugation the supernatant is filtered.

The above defined supernatant preferably comprises at least one peptide comprising the amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

More preferably, said supernatant comprises peptides comprising the amino acid sequence of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

In a preferred embodiment of the invention, the inflammatory cytokine is gamma-IFN.

The tumor cell is preferably an established tumor cell line, or a combination of tumor cell lines expressing a specific tumor antigen peptide repertoire or a tumor cell isolated by a tumor affected subject. Said subject may be human or animal, preferably human.

Preferably, the tumor cell derives from solid or non-solid tumors, including melanoma, lung carcinoma, ovarian cancer, pancreatic cancer, glioma, glioblastoma, hepatocellular carcinoma, bladder cancer, stomach cancer, colorectal adenocarcinoma, prostate adenocarcinoma, sarcoma, osteosarcoma, leukemia and T cell-lymphoma and the said specific tumor antigen peptide repertoire is specific for said tumor.

In a preferred aspect, the PRR agonists are Gram-negative, preferably belonging to the Salmonella genus, more preferably to non virulent strains of Salmonella genus, or Gram-positive bacteria or components thereof.

Preferably, gram negative bacteria components are LPS and/or flagellin or Gram positive bacteria component is Lipoteichoic acid (LTA).

A further object of the inventions is a supernatant or a fraction thereof obtainable by the method as defined above. Preferably, the supernatant or a fraction thereof comprises peptides characterized through mass Spectrometry analysis by at least one of the pics selected from the pics represented in FIGS. 3 and/or 12.

More preferably, the supernatant or fraction thereof comprises at least one peptide comprising the amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

More preferably, the supernatant or fraction thereof comprises peptides each comprising the amino acid sequence of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

A further object of the invention is an isolated peptide comprising the amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

Other objects of the invention are: an isolated nucleic acid encoding the peptide or orthologues, variants or fragments thereof as above defined; an expression vector capable of expressing a nucleic acid as above defined; an isolated host cell comprising the nucleic acid according to the invention or the expression vector as above defined, wherein said host cell preferably is an antigen presenting cell, in particular a dendritic cell or antigen presenting cell.

A further object of the invention is a method of producing a peptide as above defined, the method comprising culturing the host cell as abode defined that expresses the nucleic acid as above defined or the expression vector as above defined, and isolating the peptide from the host cell or its culture medium.

Another object of the invention is the supernatant of the invention or a fraction thereof or at least one purified peptide from said cell culture supernatant or the isolated peptide as above defined or the nucleic acid or the expression vector or the host cell as above defined used in combination with a therapeutic agent, preferably at least one antibody and/or chemotherapeutic and/or immune checkpoint inhibitor.

A further object of the invention is a specific tumor antigen peptide repertoire loaded and/or activated dendritic cell obtainable by the above method.

Preferably, said specific tumor antigen peptide repertoire loaded and/or activated dendritic cell is used in combination with a therapeutic agent, preferably at least one antibody and/or chemotherapeutic and/or immune checkpoint inhibitor.

A further object of the invention is a tumor antigen-specific CTL obtainable by the above method. Preferably, said tumor antigen-specific CTL is used in combination with a therapeutic agent, preferably at least one antibody and/or chemotherapeutic and/or immune checkpoint inhibitor.

Another object of the invention is the supernatant of the invention or a fraction thereof or at least one purified peptide from said cell culture supernatant or the isolated peptide as above defined or the nucleic acid or the expression vector or the host cell as above defined or the specific tumor antigen peptide repertoire loaded and/or activated dendritic cell as above defined or the tumor antigen-specific CTL as above defined, for use as a medicament, more preferably for use in the prevention and/or treatment of tumors and/or for use as a tumor immunotherapeutic agent or as tumor vaccine.

Another object of the invention is an immunogenic composition or vaccine comprising the supernatant as above defined or a fraction thereof and/or at least one purified peptide from said cell culture supernatant and/or at least one of the isolated peptide as above defined and/or the nucleic acid and/or the expression vector and/or the host cell and/or at least one specific tumor antigen peptide repertoire loaded and/or activated dendritic cell and/or at least one tumor antigen-specific CTL as above defined and at least one pharmaceutically acceptable carrier and/or adjuvant.

A further object of the invention is a kit comprising:

(a) a container that contains a pharmaceutical composition containing the supernatant as above defined or a fraction thereof and/or at least one purified peptide from said cell culture supernatant and/or at least one of the isolated peptide as above defined and/or the nucleic acid and/or the expression vector and/or the host cell and/or at least one specific tumor antigen peptide repertoire loaded and/or activated dendritic cell and/or at least one tumor antigen-specific CTL as described above, in solution or in lyophilized form;
(b) optionally, a second container containing a diluent or reconstituting solution for the lyophilized formulation;
(c) optionally, at least one more peptide selected from the group consisting of the peptides according to SEQ ID Nos: 2 to 32, and (d) optionally, instructions for (i) use of the solution or (ii) reconstitution and/or use of the lyophilized formulation.

Preferably said kit further comprises one or more of (iii) a buffer, (iv) a diluent, (v) a filter, (vi) a needle, or (v) a syringe.

The specific tumor antigen peptide repertoire loaded and/or activated dendritic cells or the tumor antigen-specific CTLs of the invention may be administered to a subject in suitable amounts by conventional administration routes, such as intradermal, also at multiple administration dosages, i.e. at weekly intervals, for tumor treatments.

The methods of the invention are preferably in vitro or ex vivo methods.

Said tumor cell culture preferably express said specific tumor antigen peptide repertoire. The term “expressing” includes also antigens which are not expressed by the cells but are generated by the proteasome of the tumor cell.

In a preferred embodiment of the invention the above defined peptide comprises a sequence selected from the group consisting of SEQ IN Nos. 3-10 or orthologues, variants or fragments thereof.

Any combination of two, three, four, five, . . . thirty-two peptides as above defined is comprised in the present invention.

The supernatant as above defined may also comprise the peptide of SEQ ID NO:1, or orthologues, variants or fragments thereof.

Tumor cells present specific tumor antigen peptide repertoire derived either from tumor associated antigens or by proteins expressed also in non tumor cells that are specifically cleaved in the tumor cell, as i.e. described in Mocellin S, Mandruzzato S, Bronte V, et al. Part I: Vaccines for solid tumours. Lancet Oncol 2004; 5:681-9.

An antigen loaded DC is a well known definition for the skilled person, and refers also to antigen degradation and peptide loading onto MHC molecules occurring intracellularly in Antigen Presenting Cells (APCs, such as dendritic cells). CD8+ and CD4+ T cells expressing clonally distributed receptors recognize fragments of antigens (peptides) associated with MHC class I and II molecules, respectively (Guermonprez P, Valladeau J, Zitvogel L, et al. Antigen presentation and T cell stimulation by dendritic cells. Annu Rev Immunol 2002; 20:621-67).

As to the meaning of activated DCs, a well known definition for the skilled person is that the dendritic cell matures into a highly effective presenting cell (APC) and undergoes changes that enable it to activate antigen-specific lymphocytes that it encounters i.e. in the lymph node. Activated dendritic cells secrete cytokines that influence both innate and adaptive immune responses (Immunobiology: The Immune System in Health and Disease. 5th edition. Janeway C A Jr, Travers P, Walport M, et al. New York: Garland Science; 2001).

Pattern recognition receptors (PRRs) refer to germline-encoded receptors that recognize molecular structures that are broadly shared by pathogens, known as pathogen-associated molecular patterns (PAMPs, Kawai T, Akira S. Toll-like receptors and their crosstalk with other innate receptors in infection and immunity. Immunity 2011; 34:637-50).

A PRR agonist refers to a compound (either natural or synthetic) that binds to PRR and triggers a response.

The subject to be treated may be a human or an animal.

As used herein, the term “cell culture supernatant” refers to the medium wherein at least one tumour cell exposed to at least one PRR agonist and/or inflammatory cytokine is cultured, said medium being substantially free of tumour cells, of the lysate of such cells or of fragments thereof, and which contains a mixture of tumour antigen peptides that have been secreted into the medium. The cell culture supplement preferably doesn't comprise the PRR agonist and/or inflammatory cytokine. The “cell culture supernatant” includes also fraction thereof.

Preferably, a “cell culture supernatant” will contain at least one of the secreted peptides of SEQ ID Nos. 2-32, and fragments or aggregates thereof. The cell culture supernatant of the present invention may also include other secreted proteins, such as Nischarin (Nisch), SCY1-like protein 2 (Scyl2), GrpE protein homolog 1 (Grpel1), Transcriptional activator protein Pur-beta (Purb), CLIP-associating protein 1 (Clasp1), SEC23-interacting protein (Sec23ip), Mitogen-activated protein kinase 1 (Mapk1), AP-2 complex subunit mu (Ap2m1), Ras-related protein Rab-21 (Rab21), Ras-related protein Rap-1A (Rap1a), Heat shock 70 kDa protein 4L (Hspa41), Vesicle-fusing ATPase (Nsf), Heat shock 70 kDa protein 1A (Hspa1b), Golgi phosphoprotein 3 (Golph3), Myosin-9 (Myh9).

In some instances, the cell culture supernatant may be supplemented with additional recombinant or purified secreted antigens, such as with classical tumor antigens such as MAGE-3, MelanA/Mart1, NY-ESO-1, as well as with any of the other secreted proteins.

The therapeutic agent combined with the supernatant as above defined or a fraction thereof or with at least one purified peptides from said cell culture supernatant or with the isolated peptide of the invention or with the nucleic acid or the expression vector as above defined or with the host cell of of the invention, or with the specific tumor antigen peptide repertoire loaded and/or activated dendritic cells or with the tumor antigen-specific CTL as above defined may be at least one immune checkpoint inhibitor (e.g. anti-PDLL, anti-PD1, anti-CTLA4, such as ipilimumab (Yervoy; Bristol-Myers Squibb), nivolumab (Opdivo; Bristol-Myers Squibb/Ono Pharmaceuticals), and pembrolizumab ((Keytruda; Merck & Co.), MEDI4736 (AstraZeneca) and MPDL3280A (Roche/Genentech)) (Webster R M. The immune checkpoint inhibitors: where are we now? Nature Reviews Drug Discovery 13, 883-884 (2014)).

Dendritic cells can be obtained from any source and may be autologous or allogeneic. As used herein, a cell that is “autologous” to a subject means the cell was isolated from the subject or derived from a cell that was isolated from the subject.

The term “peptide” is used herein to designate a series of amino acid residues, connected one to the other typically by peptide bonds between the alpha-amino and carbonyl groups of the adjacent amino acids. The peptides are typically 11 amino acids in length, but can be as short as 8 amino acids in length, and as long as 21 or 10, 11, 12, 13, 14, 15, 16, 17, 18, 19 or 20 amino acids in length.

The nucleic acid may be DNA, cDNA, PNA, CNA, RNA or a combination thereof.

In a preferred embodiment of the invention the peptide is not the intact human tumour associated polypeptide.

Preferably the peptides of the invention have the ability to bind to a molecule of the human major histocompatibility complex (MHC) class-I or II.

The peptide of the invention has preferably an overall length of between 9 and 100, preferably between 9 and 30, and most preferred between 9 and 16 amino acids.

As used herein, a “tumor antigen peptide repertoire loaded DC” has been contacted with the tumor cell culture supernatant under conditions that allow the DC to present peptides derived from the tumor cell supernatant in the context of MHC molecules on the cell surface.

In another preferred embodiment the vaccine is a nucleic acid vaccine. It is known that inoculation with a nucleic acid vaccine, such as a DNA vaccine, encoding a polypeptide leads to a T-cell response. It may be administered directly into the patient, into the affected organ or systemically, or applied ex vivo to cells derived from the patient or a human cell line which are subsequently administered to the patient, or used in vitro to select a subpopulation from immune cells derived from the patient, which are then re-administered to the patient. If the nucleic acid is administered to cells in vitro, it may be useful for the cells to be transfected so as to co-express immune-stimulating cytokines, such as interleukin-2 or GM-CSF. The nucleic acid vaccine may also be administered with an adjuvant such as BCG or alum. However, it is preferred if the nucleic acid vaccine is administered without adjuvant. The polynucleotide or nucleic acid may be substantially pure, or contained in a suitable vector or delivery system. Suitable vectors and delivery systems include viral, such as systems based on adenovirus, vaccinia virus, retroviruses, herpes virus, adeno-associated virus or hybrids containing elements of more than one virus. Non-viral delivery systems include cationic lipids and cationic polymers as are well known in the art of DNA delivery. Physical delivery, such as via a “gene-gun” may also be used. The peptide or peptide encoded by the nucleic acid may be a fusion protein, for example with an epitope from tetanus toxoid which stimulates CD4-positive T-cells. Suitably, any nucleic acid administered to the patient is sterile and pyrogen free. Naked DNA may be given intramuscularly or intradermally or subcutaneously. The peptides may be given intramuscularly, intradermally or subcutaneously.

Conveniently, the nucleic acid vaccine may comprise any suitable nucleic acid delivery means. The nucleic acid, preferably DNA, may be naked (i.e. with substantially no other components to be administered) or it may be delivered in a liposome or as part of a viral vector delivery system.

As used herein, an “immunogenic composition” is a composition which is capable of stimulating an immune response to one or more antigens in the composition when administered to a subject. A non-limiting example of an immunogenic composition described herein is a vaccine (e.g., a DC-based vaccine), e.g., for the treatment of cancer.

As used herein, the phrase “pharmaceutically acceptable” refers to molecular entities and compositions that are generally believed to be physiologically tolerable and do not typically produce an allergic or similar untoward reaction, such as gastric upset, dizziness and the like, when administered to a human.

The composition of the invention may contain a therapeutically effective amount of DCs.

A “therapeutically effective amount” means the amount of a compound (or, e.g., cells, e.g., DCs) that, when administered to a mammal for treating or preventing a state, disorder or condition, is sufficient to effect such treatment or prevention. The “therapeutically effective amount” will vary depending on the compound or cells, the disease and its severity and the age, weight, physical condition and responsiveness of the mammal to be treated.

The pharmaceutical composition can be chosen on the basis of the treatment requirements. Such pharmaceutical compositions according to the invention can be administered in the form of tablets, capsules, oral preparations, powders, granules, pills, injectable, or infusible liquid solutions, suspensions, suppositories, preparation for inhalation.

A reference for the formulations is the book by Remington (“Remington: The Science and Practice of Pharmacy”, Lippincott Williams & Wilkins, 2000).

The supernatant of the invention or a fraction thereof and/or at least one purified peptides from said cell culture supernatant and/or the isolated peptide and/or the nucleic acid and/or the expression vector and/or the host cell as above defined and/or the dendritic cells and/or the CTLs of the present invention may be provided in a pharmaceutical preparation. Said preparation may be employed in the treatment or prevention of cancer in an individual, virtually suffering from any type of solid and blood cancer. The DCs or the CTLs of the invention may be weekly inoculated preferentially but not exclusively intradermally, in a dose of at least 10 millions DCs or CTLs. The therapy may be made up of a number of injections comprised between 2 and 20. The product is preferentially but not exclusively stored in 10% glycerol, thawed for a max of 15′ at RT and immediately administered.

The terms “treat or treatment” and “prevent or prevention” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect, in this respect, the inventive methods can provide any amount of any level of treatment or prevention of a condition associated with inflammation, e.g. in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof. According to the present invention, an “effective amount” of a composition is one which is sufficient to achieve a desired biological effect, in this case a decrease in inflammatory response in the animal or human. It is understood that the effective dosage will be dependent upon the age, sex, health, and weight of the recipient, kind of concurrent treatment, if any, frequency of treatment, and the nature of the effect desired. The preferred dosage can be tailored to the individual subject, as is understood and determinable by one of skill in the art, without undue experimentation. Doses of e.g. between 50 pg and 1.5 mg, preferably 125 pg to 500 ig, of peptides or DNA may be given and will depend on the respective peptide or DNA. The present invention has use in human and animal health (veterinary use), preferably in Canis lupus familiaris, Felis catus, Equus caballus, Bos Taurus.

Amounts effective for a therapeutic or prophylactic use will depend on, for example, the stage and severity of the disease or disorder being treated and the judgment of the prescribing physician. The size of the dose will also be determined by the compound selected, method of administration, timing and frequency of administration as well as the existence, nature, and extent of any adverse side-effects that might accompany the administration of a particular compound and the desired physiological effect. It will be appreciated by one of skill in the art that various diseases or disorders could require prolonged treatment involving multiple administrations, perhaps using the compound of the invention in each or various rounds of administration. The disclosed compounds can be administered in a composition (e.g., pharmaceutical composition) that can comprise at least one excipient (e.g., a pharmaceutically acceptable excipient), as well as other therapeutic agents (e.g., anti-inflammatory agents). The composition can be administered by any suitable route, including parenteral, topical, oral, or local administration. The pharmaceutically acceptable excipient is preferably one that is chemically inert to the compounds above disclosed and one that has little or no side effects or toxicity under the conditions of use. Such pharmaceutically acceptable carriers include, but are not limited to, water, saline, Cremophor EL (Sigma Chemical Co., St. Louis, Mo.), propylene glycol, polyethylene glycol, alcohol, and combinations thereof. The choice of carrier will be determined in part by the particular compound as well as by the particular method used to administer the composition. Accordingly, there is a wide variety of suitable formulations of the composition. The pharmaceutical composition in the context of an embodiment of the invention can be, for example, in the form of a pill, capsule, or tablet, each containing a predetermined amount of one or more of the active compounds and preferably coated for ease of swallowing, in the form of a powder or granules, or in the form of a solution or suspension. For oral administration, fine powders or granules may contain diluting, dispersing, and or surface active agents and may be present, for example, in water or in a syrup, in capsules or sachets in the dry state, or in a nonaqueous solution or suspension wherein suspending agents may be included, or in tablets wherein binders and lubricants may be included. Components such as sweeteners, flavoring agents, preservatives (e.g., antimicrobial preservatives), suspending agents, thickening agents, and/or emulsifying agents also may be present in the pharmaceutical composition. When administered in the form of a liquid solution or suspension, the formulation can contain one or more of the active compounds and purified water. Optional components in the liquid solution or suspension include suitable preservatives (e.g., antimicrobial preservatives), buffering agents, solvents, and mixtures thereof. A component of the formulation may serve more than one function. Preservatives may be used. Suitable preservatives may include, for example, methylparaben, propylparaben, sodium benzoate, and benzalkonium chloride. A mixture of two or more preservatives optionally may be used. The preservative or mixtures thereof are typically present in an amount of about 0.0001% to about 2% by weight of the total composition. Suitable buffering agents may include, for example, citric acid, sodium citrate, phosphoric acid, potassium phosphate, and various other acids and salts. A mixture of two or more buffering agents optionally may be used. The buffering agent or mixtures thereof are typically present in an amount of about 0.001% to about 4% by weight of the total composition. The following formulations for oral, aerosol, parenteral (e.g., subcutaneous, intravenous, intraarterial, intramuscular, intradermal, interperitoneal, and intrathecal), and rectal administration are merely exemplary and are in no way limiting. Formulations suitable for oral administration can consist of (a) liquid solutions, such as an effective amount of the compound dissolved in diluents, such as water, saline, or orange juice; (b) capsules, sachets, tablets, lozenges, and troches, each containing a predetermined amount of the active ingredient, as solids or granules; (c) powders; (d) suspensions in an appropriate liquid; and (e) suitable emulsions. Liquid formulations may include diluents, such as water and alcohols, for example, ethanol, benzyl alcohol, and the polyethylene alcohols, either with or without the addition of a pharmaceutically acceptable surfactant, suspending agent, or emulsifying agent. Capsule forms can be of the ordinary hard- or soft-shelled gelatin type containing, for example, surfactants, lubricants, and inert fillers, such as lactose, sucrose, calcium phosphate, and cornstarch. Tablet forms can include one or more of lactose, sucrose, mannitol, corn starch, potato starch, alginic acid, microcrystalline cellulose, acacia, gelatin, guar gum, colloidal silicon dioxide, croscarmellose sodium, talc, magnesium stearate, calcium stearate, zinc stearate, stearic acid, and other excipients, colorants, diluents, buffering agents, disintegrating agents, moistening agents, preservatives, flavoring agents, and pharmacologically compatible carriers. Lozenge forms can comprise the active ingredient in a flavor, usually sucrose and acacia or tragacanth, as well as pastilles comprising the active ingredient in an inert base, such as gelatin and glycerin, or sucrose and acacia, emulsions, gels, and the like containing, in addition to the active ingredient, such carriers as are known in the art. The above compounds, alone or in combination with other suitable components, can be made into aerosol formulations to be administered via inhalation. These aerosol formulations can be placed into pressurized acceptable propellants, such as dichlorodifluoromethane, propane, nitrogen, and the like. They also may be formulated as pharmaceuticals for non-pressured preparations, such as in a nebulizer or an atomizer. Formulations suitable for parenteral administration include aqueous and nonaqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The above compounds may be administered in a physiologically acceptable diluent in a pharmaceutical carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol, glycerol ketals, such as 2,2-dimethyl-1,3-dioxolane-4-methanol, ethers, such as poly(ethyleneglycol) 400, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants. Oils, which can be used in parenteral formulations, include petroleum, animal, vegetable, or synthetic oils. Specific examples of oils include peanut, soybean, sesame, cottonseed, corn, olive, petrolatum, and mineral. Suitable fatty acids for use in parenteral formulations include oleic acid, stearic acid, and isostearic acid. Ethyl oleate and isopropyl myristate are examples of suitable fatty acid esters.

Suitable soaps for use in parenteral formulations may include fatty alkali metal, ammonium, and triethanolamine salts, and suitable detergents include (a) cationic detergents such as, for example, dimethyl dialkyl ammonium halides, and alkyl pyridinium halides, (b) anionic detergents such as, for example, alkyl, aryl, and olefin sulfonates, alkyl, olefin, ether, and monoglyceride sulfates, and sulfosuccinates, (c) nonionic detergents such as, for example, fatty amine oxides, fatty acid alkanolamides, and polyoxyethylene-polypropylene copolymers, (d) amphoteric detergents such as, for example, alkyl-beta-aminopropionates, and 2-alkyl-imidazoline quaternary ammonium salts, and (3) mixtures thereof.

Suitable preservatives and buffers can be used in such formulations. In order to minimize or eliminate irritation at the site of injection, such compositions may contain one or more nonionic surfactants having a hydrophile-lipophile balance (HLB) of from about 12 to about 17. The quantity of surfactant in such formulations ranges from about 5% to about 15% by weight. Suitable surfactants include polyethylene sorbitan fatty acid esters, such as sorbitan monooleate and the high molecular weight adducts of ethylene oxide with a hydrophobic base, formed by the condensation of propylene oxide with propylene glycol. The parenteral formulations can be presented in unit-dose or multi-dose sealed containers, such as ampoules and vials, and can be stored in a freeze-dried (lyophilized) condition requiring only the addition of the sterile liquid carrier, for example, water, for injections, immediately prior to use. Extemporaneous injection solutions and suspensions can be prepared from sterile powders, granules, and tablets. The above compounds may be administered as an injectable formulation. The requirements for effective pharmaceutical carriers for injectable compositions are well known to those of ordinary skill in the art. See Pharmaceutics and Pharmacy Practice, J. B. Lippincott Co., Philadelphia, Pa., Banker and Chalmers, eds., pages 238-250 (1982), and ASHP Handbook on Injectable Drugs, Toissel, 4th ed., pages 622-630 (1986). Topical formulations, including those that are useful for transdermal drug release, are well known to those of skill in the art and are suitable in the context of embodiments of the invention for application to skin. The concentration of a compound of embodiments of the invention in the pharmaceutical formulations can vary, e.g., from less than about 1%, usually at or at least about 10%, to as much as 20% to 50% or more by weight, and can be selected primarily by fluid volumes, and viscosities, in accordance with the particular mode of administration selected. Methods for preparing administrable (e.g., parenterally administrable) compositions are known or apparent to those skilled in the art and are described in more detail in, for example, Remington's Pharmaceutical Science (17th ed., Mack Publishing Company, Easton, Pa., 1985). In addition to the aforedescribed pharmaceutical compositions, the above compounds can be formulated as inclusion complexes, such as cyclodextrin inclusion complexes, or liposomes. Liposomes can serve to target the compounds to a particular tissue. Many methods are available for preparing liposomes, as described in, for example, Szoka et al., Ann. Rev. Biophys. Bioeng., 9:467 (1980) and U.S. Pat. Nos. 4,235,871, 4,501,728, 4,837,028, and 5,019,369.

Preferably, the compound as above described may be formulated for oral or local administration in a sustained or controlled release acid resistant delivery system.

When the agent of the invention is administered with one or more additional therapeutic agents, one or more additional therapeutic agents can be coadministered to the mammal. By “coadministering” is meant administering one or more additional therapeutic agents and the above compound sufficiently close in time such that the compound can enhance the effect of one or more additional therapeutic agents. In this regard, the compound can be administered first and the one or more additional therapeutic agents can be administered second, or vice versa. Alternatively, the compound and the one or more additional therapeutic agents can be administered simultaneously. The delivery systems useful in the context of embodiments of the invention may include time-released, delayed release, and sustained release delivery systems such that the delivery of the inventive composition occurs prior to, and with sufficient time to cause, sensitization of the site to be treated. The inventive composition can be used in conjunction with other therapeutic agents or therapies. Such systems can avoid repeated administrations of the inventive composition, thereby increasing convenience to the subject and the physician, and may be particularly suitable for certain composition embodiments of the invention. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are lipids including sterols such as cholesterol, cholesterol esters, and fatty acids or neutral fats such as mono-di-and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active composition is contained in a form within a matrix such as those described in U.S. Pat. Nos. 4,452,775, 4,667,014, 4,748,034, and 5,239,660 and (b) diffusional systems in which an active component permeates at a controlled rate from a polymer such as described in U.S. Pat. Nos. 3,832,253 and 3,854,480. In addition, pump-based hardware delivery systems can be used, some of which are adapted for implantation. In order to increase the shelf-life of the composition according to the present the cell culture supernatant may be lyophilised. Methods for lyophilising such preparations are well known to the person skilled in the art.

According to a preferred embodiment of the present invention the administration of the product can be accompanied by the administration of immunostimmulatory agents and/or adjuvants. Its usage is as well suitable in concomitance with chemotherapy as well as during the pauses between chemotherapic cycles.

The pharmaceutical compositions as above defined may be administered in a single dosage.

The expert in the art will select the form of administration and effective dosages by selecting suitable diluents, adjuvants and/or excipients.

The exposure of the tumor cell culture to the PRR or inflammatory cytokine may be obtained with any methods known to the skilled man. The suitable conditions of the exposure will be chosen so that the opening of CxH is increased.

In a preferred embodiment the connexins are connexin 43 (encoded e.g. by Mus musculus: gene ID: 14609; Homo sapiens: gene ID: 2697) and/or connexin 40 (encoded e.g. by Mus musculus gene ID: 14613; Homo sapiens gene ID: 2702), and/or connexin 45, (encoded e.g. by Mus musculus gene ID: 14615; Homo sapiens gene ID: 10052) and/or connexin 47 (encoded e.g. by Mus musculus gene ID: 118454; Homo sapiens gene ID: 57165), and/or connexin 50 (encoded e.g. by Mus musculus gene ID: 14616 and Homo sapiens: gene ID: 2703) or orthologous, allelic variants or iso forms thereof.

As used herein, the term “orthologous” refers to protein or peptides in species different with respect to the peptides of SEQ ID Nos. 1-32 in Mus Musculus. As an example of said orthologous, the corresponding proteins or peptides in Canis lupus familiaris, Felis catus, Equus caballus, Bos Taurus Rattus norvegicus, Gallus gallus, Xenopus laevis and Danio rerio can be cited.

The term “fraction thereof” referred to the supernatant, also includes at least one peptide purified from the supernatant.

The term “fragment,” when referring to a coding sequence, means a portion of DNA comprising less than the complete coding region, whose expression product retains essentially the same biological function or activity as the expression product of the complete coding region.

The term “active fragment” or “functional fragment” means a fragment that generates an immune response (i.e., has immunogenic activity) when administered, alone or optionally with a suitable adjuvant, to an animal, such as a mammal, for example, a rabbit or a mouse, and also including a human, such immune response taking the form of stimulating a T-cell response within the recipient animal, such as a human. Alternatively, the “active fragment” may also be used to induce a T-cell response in vitro.

As used herein, the terms “portion,” “segment,” and “fragment,” when used in relation to polypeptides or peptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide. This means that any such fragment will necessarily contain as part of its amino acid sequence a segment, fragment or portion, that is substantially identical, if not exactly identical, to a sequence of SEQ ID NO: 1 to 32, which correspond to the naturally occurring, or “parent” proteins of the SEQ ID NO: 1 to 32. When used in relation to polynucleotides, such terms refer to the products produced by treatment of said polynucleotides with any of the common endonucleases. By a “variant” of the given amino acid sequence the inventors mean that the side chains of, for example, one or two of the amino acid residues are altered (for example by replacing them with the side chain of another naturally occurring amino acid residue or some other side chain) such that the peptide is still able to bind to an HLA molecule in substantially the same way as a peptide consisting of the given amino acid sequence in SEQ ID NO:1 to 32. For example, a peptide may be modified so that it at least maintains, if not improves, the ability to interact with and bind to the binding groove of a suitable MHC molecule, such as HLA-A*02 or -DR, and in that way it at least maintains, if not improves, the ability to bind to the TCR of activated CTL. These CTL can subsequently cross-react with cells and kill cells that express a polypeptide which contains the natural amino acid sequence of the cognate peptide as defined in the aspects of the invention. As can be derived from the scientific literature (Rammensee et al., 1997) and databases (Rammensee et al., 1999), certain positions of HLA binding peptides are typically anchor residues forming a core sequence fitting to the binding motif of the HLA receptor, which is defined by polar, electrophysical, hydrophobic and spatial properties of the polypeptide chains constituting the binding groove. Thus one skilled in the art would be able to modify the amino acid sequences set forth in SEQ ID NO: 1 to 32, by maintaining the known anchor residues, and would be able to determine whether such variants maintain the ability to bind MHC class I or II molecules. The variants of the present invention retain the ability to bind to the TCR of activated CTL, which can subsequently cross-react with—and kill cells that express a polypeptide containing the natural amino acid sequence of the cognate peptide as defined in the aspects of the invention.

Those amino acid residues that do not substantially contribute to interactions with the T-cell receptor can be modified by replacement with another amino acid whose incorporation does not substantially affect T-cell reactivity and does not eliminate binding to the relevant MHC. Thus, apart from the proviso given, the peptide of the invention may be any peptide (by which term the inventors include oligopeptide or polypeptide), which includes the amino acid sequences or a portion or variant thereof as given. “Percent (%) amino acid sequence identity” or “Percent identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

In a preferred embodiment the variant of the peptide according to the invention, is at least 85% homologous to SEQ ID NO: 1 to SEQ ID NO: 32 and/or will induce T cells cross-reacting with said peptide.

In the present invention, the term “homologous” refers to the degree of identity between sequences of two amino acid sequences, i.e. peptide or polypeptide sequences. The aforementioned “homology” is determined by comparing two sequences aligned under optimal conditions over the sequences to be compared. The sequences to be compared herein may have an addition or deletion (for example, gap and the like) in the optimum alignment of the two sequences. Such a sequence homology can be calculated by creating an alignment using, for example, the ClustalW algorithm (Nucleic Acid Res., 22(22): 4673 4680 (1994). Commonly available sequence analysis software, more specifically, Vector NTI, GENETYX or analysis tools provided by public databases. A person skilled in the art will be able to assess, whether T cells induced by a variant of a specific peptide will be able to cross-react with the peptide itself (Fong et al., 2001); (Zaremba et al., 1997; Colombetti et al., 2006; Appay et al., 2006).

The expression “peptide” is intended to include also the corresponding peptide encoded from an orthologous or homologous genes, functional mutants, functional derivatives, functional fragments or analogues, isoforms thereof.

In the present invention “functional mutants” of the peptides are peptides that may be generated by mutating one or more amino acids in their sequences and that maintain their activity e.g. immunogenicity. Indeed, the polypeptide of the invention, if required, can be modified in vitro and/or in vivo, for example by glycosylation, myristoylation, amidation, carboxylation or phosphorylation, and may be obtained, for example, by synthetic or recombinant techniques known in the art.

In the present invention “functional” is intended for example as “maintaining their activity” e.g. maintaining immunogenicity or the ability of inducing an antitumor immune response.

The term “analogue” as used herein referring to a peptide means a modified peptide wherein one or more amino acid residues of the peptide have been substituted by other amino acid residues and/or wherein one or more amino acid residues have been deleted from the peptide and/or wherein one or more amino acid residues have been added to the peptide. Such addition or deletion of amino acid residues can take place at the N-terminal of the peptide and/or at the C-terminal of the peptide.

The term “derivative” as used herein in relation to a protein means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like. As used herein, the term “derivatives” also refers to longer or shorter peptides having e.g. a percentage of identity of at least 41%, preferably at least 41.5%, 50%, 54.9%, 60%, 61.2%, 64.1%, 65%, 70% or 75%, more preferably of at least 85%, as an example of at least 90%, and even more preferably of at least 95% with the peptide of the invention, or with an amino acid sequence of the correspondent region encoded from the peptide orthologous or homologous gene.

The original peptides disclosed herein can be modified by the substitution of one or more residues at different, possibly selective, sites within the peptide chain, if not otherwise stated. Such substitutions may be of a conservative nature, for example, where one amino acid is replaced by an amino acid of similar structure and characteristics, such as where a hydrophobic amino acid is replaced by another hydrophobic amino acid. Even more conservative would be replacement of amino acids of the same or similar size and chemical nature, such as where leucine is replaced by isoleucine. In studies of sequence variations in families of naturally occurring homologous proteins, certain amino acid substitutions are more often tolerated than others, and these are often show correlation with similarities in size, charge, polarity, and hydrophobicity between the original amino acid and its replacement, and such is the basis for defining “conservative substitutions.” Conservative substitutions are herein defined as exchanges within one of the following five groups: Group 1-small aliphatic, nonpolar or slightly polar residues (Ala, Ser, Thr, Pro, Gly); Group 2-polar, negatively charged residues and their amides (Asp, Asn, Glu, Gln); Group 3 polar, positively charged residues (His, Arg, Lys); Group 4—large, aliphatic, nonpolar residues (Met, Leu, Ile, Val, Cys); and Group 5-large, aromatic residues (Phe, Tyr, Trp).

Less conservative substitutions might involve the replacement of one amino acid by another that has similar characteristics but is somewhat different in size, such as replacement of an alanine by an isoleucine residue. Highly non-conservative replacements might involve substituting an acidic amino acid for one that is polar, or even for one that is basic in character. Such “radical” substitutions cannot, however, be dismissed as potentially ineffective since chemical effects are not totally predictable and radical substitutions might well give rise to serendipitous effects not otherwise predictable from simple chemical principles.

Of course, such substitutions may involve structures other than the common L-amino acids. Thus, D-amino acids might be substituted for the L-amino acids commonly found in the antigenic peptides of the invention and yet still be encompassed by the disclosure herein. In addition, amino acids possessing non-standard R groups (i.e., R groups other than those found in the common 20 amino acids of natural proteins) may also be used for substitution purposes to produce immunogens and immunogenic polypeptides according to the present invention.

If substitutions at more than one position are found to result in a peptide with substantially equivalent or greater antigenic activity as defined below, then combinations of those substitutions will be tested to determine if the combined substitutions result in additive or synergistic effects on the antigenicity of the peptide. At most, no more than 4 positions within the peptide would simultaneously be substituted.

In the context of the present invention the term “tumor” includes solid or non-solid tumors, comprising melanoma, lung carcinoma, ovarian cancer, pancreatic cancer, glioma, glioblastoma, hepatocellular carcinoma, bladder cancer, stomach cancer, colorectal adenocarcinoma, prostate adenocarcinoma, sarcoma, osteosarcoma, leukemia and T cell-lymphoma.

A further object of the invention is a method of treating and/or preventing a tumour or metastasis comprising administering a therapeutically effective amount of a compound of the invention (i.e. the supernatant of the invention or a fraction thereof and/or at least one purified peptide from said cell culture supernatant and/or at least one of the isolated peptide of the invention and/or the nucleic acid of and/or the expression vector and/or the host cell and/or at least one specific tumor antigen peptide repertoire loaded and/or activated dendritic cell and/or at least one tumor antigen-specific CTL according to the invention and/or viral particle as above defined).

The method for treating or preventing a cancer or metastasis, comprises administering to a patient in need thereof an effective amount of the compound of the invention as above defined.

In some aspects, the invention comprises a method for treating or preventing cancer or metastasis in a subject, the method comprising administering to a subject in need thereof an effective amount of the compound as above defined simultaneously or sequentially with an a therapeutic agent, e.g. at least one antibody and/or chemotherapeutic and/or immune checkpoint inhibitor.

A further object of the invention is an in vitro method for producing activated cytotoxic T lymphocytes (CTL), the method comprising contacting in vitro CTL with antigen loaded human class I or II MHC molecules expressed on the surface of a suitable antigen-presenting cell or an artificial construct mimicking an antigen-presenting cell for a period of time sufficient to activate said CTL in an antigen specific manner, wherein said antigen is a peptide according to the invention.

Another object of the invention is an activated cytotoxic T lymphocyte (CTL), produced by the method as above defined, that selectively recognises a cell which aberrantly expresses a polypeptide comprising an amino acid sequence selected from the group consisting of SEQ ID NO:2-32.

A further object of the invention is a method for killing target cells in a patient which target cells aberrantly express a polypeptide comprising an amino acid sequence given above, the method comprising administering to the patient an effective number of cytotoxic T lymphocytes (CTL) as above defined.

The host cell can be either prokaryotic or eukaryotic. Bacterial cells may be preferred prokaryotic host cells in some circumstances and typically are a strain of E. coli such as, for example, the E. coli strains DH5 available from Bethesda Research Laboratories Inc., Bethesda, Md., USA, and RR1 available from the American Type Culture Collection (ATCC) of Rockville, Md., USA (No ATCC 31343). Preferred eukaryotic host cells include yeast, insect and mammalian cells, preferably vertebrate cells such as those from a mouse, rat, monkey or human fibroblastic and colon cell lines. Yeast host cells include YPH499, YPH500 and YPH501, which are generally available from Stratagene Cloning Systems, La Jolla, Calif. 92037, USA. Preferred mammalian host cells include Chinese hamster ovary (CHO) cells available from the ATCC as CCL61, NIH Swiss mouse embryo cells NIH/3T3 available from the ATCC as CRL 1658, monkey kidney-derived COS-1 cells available from the ATCC as CRL 1650 and 293 cells which are human embryonic kidney cells. Preferred insect cells are Sf9 cells which can be transfected with baculovirus expression vectors. An overview regarding the choice of suitable host cells for expression can be found in, for example, the textbook of Paulina Balbis and Argelia Lorence “Methods in Molecular Biology Recombinant Gene Expression, Reviews and Protocols,” Part One, Second Edition, ISBN 978-1-58829-262-9, and other literature known to the person of skill.

The host cell is preferably selected in the group consisting of: bacterial cells, fungal cells, insect cells, animal cells, and plant cells, preferably said host cells is an animal cell, more preferably a human cell. In the present invention the vector is preferably an expression vector, more preferably selected in the group consisting of: plasmids, viral particles and phages.

As used herein, the term “vector” refers to an expression vector, and may be for example in the form of a plasmid, a viral particle, a phage, etc. Such vectors may include bacterial plasmids, phage DNA, baculovirus, yeast plasmids, vectors derived from combinations of plasmids and phage DNA, viral DNA such as vaccinia, adenovirus, fowl pox virus, and pseudorabies. Large numbers of suitable vectors are known to those of skill in the art and are commercially available. The following vectors are provided by way of example. Bacterial: pQE70, pQE60, pQE-9 (QIAGEN), pbs, pDIO, phagescript, psiXl74, pbluescript SK, pbsks, pNH8A, pNHl[beta]a, pNH18A, pNH46A (STRATAGENE), ptrc99a, pKK223-3, pKK233-3, pDR540, pRIT5 (PHARMACIA). Eukaryotic: pWLNEO, pSV2CAT, pOG44, pXT1, pSG (STRATAGENE), pSVK3, pBPV, pMSG, pSVL (PHARMACIA). However, any other vector may be used as long as it is replicable and viable in the host. The polynucleotide sequence, preferably the DNA sequence in the vector is operatively linked to an appropriate expression control sequence(s) (promoter) to direct mRNA synthesis. As representative examples of such promoters, one can mention prokaryotic or eukaryotic promoters such as CMV immediate early, HSV thymidine kinase, early and late SV40, LTRs from retrovirus, and mouse metallothionein-I. The expression vector also contains a ribosome binding site for translation initiation and a transcription vector. The vector may also include appropriate sequences for amplifying expression. In addition, the vectors preferably contain one or more selectable marker genes to provide a phenotypic trait for selection of transformed host cells such as dihydro folate reductase or neomycin resistance for eukaryotic cell culture, or such as tetracycline or ampicillin resistance in E. coli. The host cell according to the invention may be also defined as “host cell genetically engineered”.

As used herein, the term “host cell genetically engineered” relates to host cells which have been transduced, transformed or transfected with the polynucleotide or with the vector described previously. As representative examples of appropriate host cells, one can cite bacterial cells, such as E. coli, Streptomyces, Salmonella typhimurium, fungal cells such as yeast, insect cells such as Sf9, animal cells such as CHO or COS, plant cells, etc. The selection of an appropriate host is deemed to be within the scope of those skilled in the art from the teachings herein. Preferably, said host cell is an animal cell, and most preferably a human cell. The introduction of the polynucleotide or of the vector described previously into the host cell can be effected by method well known from one of skill in the art such as calcium phosphate transfection, DEAE-Dextran mediated transfection, or electroporation.

The polynucleotide may be a vector such as for example a viral vector. Another object of the invention is a composition comprising a transformed host cell expressing a peptide selected from the peptide of SEQ ID NO: 1-32.

The man skilled in the art is well aware of the standard methods for incorporation of a polynucleotide into a host cell, for example transfection, lipofection, electroporation, microinjection, viral infection, thermal shock, transformation after chemical permeabilisation of the membrane or cell fusion.

Transformation of appropriate cell hosts with a DNA construct of the present invention is accomplished by well known methods that typically depend on the type of vector used. With regard to transformation of prokaryotic host cells, see, for example, Cohen et al (1972) Proc. Nati. Acad. Sci. USA 69, 2110, and Sambrook et al (1989) Molecular Cloning, A Laboratory Manual, Cold Spring Harbor Laboratory, Cold Spring Harbor, N.Y. Transformation of yeast cells is described in Sherman et al (1986) Methods In Yeast Genetics, A Laboratory Manual, Cold Spring Harbor, N.Y. The method of Beggs (1978) Nature 275, 104-109 is also useful. With regard to vertebrate cells, reagents useful in transfecting such cells, for example calcium phosphate and DEAE-dextran or liposome formulations, are available from Stratagene Cloning Systems, or Life Technologies Inc., Gaithersburg, Md. 20877, USA. Electroporation is also useful for transforming and/or transfecting cells and is well known in the art for transforming yeast cell, bacterial cells, insect cells and vertebrate cells.

Successfully transformed cells, i.e. cells that contain a DNA construct of the present invention, can be identified by well known techniques such as PCR. Alternatively, the presence of the protein in the supernatant can be detected using antibodies.

It will be appreciated that certain host cells of the invention are useful in the preparation of the peptides of the invention, for example bacterial, yeast and insect cells. However, other host cells may be useful in certain therapeutic methods. For example, antigen-presenting cells, such as dendritic cells, may usefully be used to express the peptides of the invention such that they may be loaded into appropriate MHC molecules. Thus, the current invention provides a host cell comprising a nucleic acid or an expression vector according to the invention.

In a preferred embodiment the host cell is an antigen presenting cell, in particular a dendritic cell or antigen presenting cell.

The present invention will be described through non-limitative examples, with reference to the following figures:

### FIGURE LEGENDS

FIG. 1. Cx43 expression in B16 cells. “-” are cells not infected. “AT” is the attenuated strain of Salmonella thyphimurium SL3261AT. Vinculin is used as loading control.

FIG. 2. Measure of ATP extracellular concentration from uninfected or infected B16 (“AT”) and B16 OVA cells treated or not with the gap junction blocker heptanol.

FIG. 3. Identification of murine endogenous peptide release into the SN by Mass Spectrometry

A) IFN-gamma secretion by OT-I CD8 T cells activated by D1 dendritic cells loaded with the indicated SN derived fractions
B) Full nLC-ESI spectrum [300-1650 Da] at 53.3 min of B16 OVA-derived SUP. JUNG (sequence: SIINFEKL [SEQ ID NO:1]) is mostly detected as double charge m/z=482.28 z=2. C) nLC-ESI-MS/MS spectrum confirmed JUNG identity (m/z 482.28, z=+2).

FIG. 4. Evaluation of stability of HLA molecules on the surface of T2 cells incubated with supernatant obtained from uninfected or infected SK-mel 24 and HT29 cells treated or not with heptanol. Stabilization of MHC class I complex on the surface of T2 cells indicate the presence of exogenous peptides in supernatant obtained from uninfected or infected SK-mel 24 and HT29 cells treated or not with heptanol. Control: T2 incubated with peptide Mart-1 (26-35); MFI is the mean of fluorescence intensity; *p<0.05, **p<0.01, ***p<0.001.

FIG. 5. IL-2 production after coculture of OVA-specific T cells with murine DCs loaded with supernatant (SN) obtained from infected (AT) or not B16 or B16 OVA cells. Negative Control: DCs (D1) alone and OVA-specific T cells alone (B3Z). Positive control: DCs loaded with OVA peptide (257-264).

FIG. 6. Cx43 expression in SK-mel24. TY means Salmonella TY21a-infected cells.

FIG. 7. a) IFN-gamma production after coculture of CTLs Mart-1 specific with moDCs loaded with supernatant obtained from SK-mel24 or 624.38 treated as indicated. Control: moDCs loaded with Mart-1 (26-35). b) Same as in a) but dendritic cells are purified ex-vivo (PDC, Plasmacytoid dendritic cells).

FIG. 8. Mice were immunized in CFA (complete Freund's adjuvant) with the indicated products. as indicated. In figure A immunization was performed with OVA protein, B16 OVA extract, supernatant (SN) from B16 OVA cells in absence or presence of the gap junction inhibitor heptanol. In figure B immunization was performed with OVA protein, SN from B16 OVA or SN from Salmonella infected B16 OVA (B16 OVA AT). After 7-9 days lymph nodes cells were re-stimulated in vitro with PPD (Tuberculin purified protein derivate) or OVA protein and IFN-gamma secretion was measured after 72 hours of culture. *p<0.05.

FIG. 9. Antibody response to Salmonella SL3261AT peptides (A) and B16 tumor peptides (B). MG132 is a proteasome inhibitor; IFA is Freund's incomplete adjuvant.

FIG. 10. Tumor growth in mice vaccinated with DCs loaded with supernatant from uninfected (B16) or Salmonella infected B16 cells (B16 AT). DCs loaded with supernatant vaccine are protective against melanoma. Control: DCs not loaded (D1 alone). *p<0.05.

FIG. 11. Tumor released peptides combined with CpG boost a stronger antitumor response. Kaplan-Meier survival curves of C57/6J mice (n=5 animals per group) vaccinated with: (A-C) increasing doses of peptides derived by Salmonella treated B16 cells (0.5×10⁶ cells (Vax1), 2×10⁶ cells (Vax2), 5×10⁶ cells (Vax3)) combined with IFA Aldara; (D) dendritic cells loaded with Vax2; (E) Vax2 mixed up with CpG-ODN1826; (F) IFA Aldara Vax2 following gavages at day −4 and −1 of 109 cfu of 1.plantarum. *P<0.05, versus adjuvant control. (G) Difference (calculated as %) between AUC derived by the mean-tumor-growth curve of Vax-treated mice and AUC derived by the mean tumor growth curve of the linked adjuvant control (H) Specific T cell degranulation (CD8+CD107a+) in a CD107a mobilization assay. Data are mean±s.e.m. of three replicates (n=5 animals per group). ***P<0.001.

FIG. 12. Mass Spectrometry analysis of murine endogenous peptides differentially released into SN of B16 Salmonella infected cells. A) Full MS spectrogram of a fraction of bacteria treated B16 cells-derived-supernatant. B) Venn Diagram resulted by the analysis of the protein associated to the detected peptides comparing bacteria treated versus untreated B16 cells-derived-supernatant.

FIG. 13. HeatMap that shows 31 identified peptides significantly more abundant in Vax supernatants; analysis were performed with MaxQuant software on the identified peptides. P value<0.05. Red color: up-regulated. Green color: down-regulated.

FIG. 14. Canine osteosarcoma cell culture A) OSA cells analyzed by optical microscope, B) OSA cells stained red for phalloidin (cytoplasmic staining) and blue with DAPI (nuclear staining), C) alkaline phosphatase staining.

FIG. 15. Cx43 expression in canine osteosarcoma cells (OSA), infected or not with Salmonella Ty21a.

### EXAMPLE

### Material and Methods

### Mice, Cell Lines and Bacterial Strain

Six-week-old female C57BL/6J mice were purchased from Charles River and maintained in specific pathogen-free animal house. The cell lines used in present study were murine melanoma B16F10 (called throughout B16) were cultured in RPMI 1640 medium supplemented with 10% fetal serum bovine, 2 mM Glutamine, 100 U/ml Penicillin, 100 μm/mL Streptomycin and 50 μM 2-Mercaptoethanol (complete RPMI).

The murine melanoma B16F10-OVA (called throughout B16 OVA) were cultured in Iscove's Modified Dulbecco's Medium (IMDM) supplemented with 10% fetal serum bovine, 2 mM Glutamine, 100 U/ml Penicillin, 100 μg/mL Streptomycin and 50 μM 2-Mercaptoethanol, 100 μg/mL Hygromycin B. Primary canine osteosarcoma cells were obtained from dissociation of dog's osteosarcoma specimens. Tissues were minced with a scalper in a cell strainer. Cells were washed with DMEM (supplemented with 10% FBS, 2 mM L-Glutamine, 100 U/mL Penicillin, 100 mg/mL Streptomycin) and filtered with a cell strainer (100 μM).

The cellular suspension was centrifuged at 300 g for 10 minutes and resuspendend with 2 mL of Lysis Buffer in order to lyse red blood cells; then the cells were washed twice with complete medium and cultured. Murine dendritic cells (D1), derived from bone marrows of C57BL/6J mice were cultured in IMDM containing 10% FBS, supplemented with 30% supernatant from granulocyte macrophage colony-stimulating factor-producing NIH-3T3 cells.

The B3Z T-cells hybridoma specific for the H-2Kb restricted OVA peptide were grown in Iscove's modified Dulbecco's medium (IMDM) supplemented with 5% FBS.

C57BL/6J OT-I mice contain transgenic T cell receptor designed to recognize OVA(₂₅₇₋₂₆₄) in the context of H2Kb. Purified CD8 OT-I T cell were re-stimulated in vitro by D1 dendritic cell line previously loaded with OVA(₂₅₇₋₂₆₄) or SN of B16 OVA infected (AT) or not, in RPMI 1640 medium supplemented with 10% fetal serum bovine, 2 mM Glutamine, 100 U/ml Penicillin, 100 μg/mL Streptomycin.

T2 cells are an HLA-A0201 hybrid human cell line lacking TAP-2 and express low amount of MHC class I on their surface. These cells were cultured in RPMI 1640 medium supplemented with 10% fetal serum bovine, 2 mM Glutamine, 100 U/ml Penicillin, 100 μg/mL Streptomycin.

The bacteria, Salmonella typhimurium SL3261AT is an aroA metabolically defective strain on SL1344 background and is grown at 37° C. in Luria broth (LB).

Vivofit® (Thyphoid vaccine live oral Ty21a) is a live attenuated vaccine containing the attenuated strain of Salmonella enteric serovar thyphi Ty21a and is grown at 37° C. in Luria broth (LB).

In Vitro Infection with Bacteria and Supernatant Production

Single bacterial colonies were grown overnight and restarted the next day to reach an absorbance at 600 nm ranging between 0.550 and 0.650 corresponding to 0,550-0,650×10₉ colony—forming units (CFUs)/mL. Tumor cells were incubated with or without Gap Junctions blocker, Heptanol (1 mM) for 1 hour and half at 37° C. Then, cells were incubated with bacteria for 1 hour and half at 37° C. at a cell-to-bacteria ratio of 1:50, in the appropriate medium without antibiotics. After incubation, the cells were washed and incubated at 37° C. in medium supplemented with gentamicin (50 μm/mL) for 2 hours. The cells were washed twice and incubated overnight with medium supplemented only with gentamicin in order to kill extracellular bacteria. The next day, the cells were centrifuged at 2000 rpm for 5 minutes; the supernatant was collected and filtered with 70 μM cell strainer. The supernatant was freeze-dried.

### Mart-1-Specific CTL Generation

To generate peptide-specific CTLs from PBMC, 106 PBMC HLA-A2+ were cultured in 1 mL of RPMI 1640 medium (supplemented with 5% human serum, 2 mM L-Glutamine, 100 U/mL Penicillin, 100 μg/mL Streptomycin, 10 μg/mL Gentamicin, 10 μg/mL beta-mercaptoethanol and 1% nonessential amino acids) in 24-well-plates. To this, 2×106 Mart-1-pulsed and irradiated (10 Gy) PBMC (HLA-A2) were added as antigen presenting cells in the same medium supplemented with 100 U/mL IL-2. To pulse PBMC they were incubated for 90 minutes at 37° C. in RPMI supplemented with 50 μM of peptide. After incubation, cell were washed twice and irradiated before mixing with the responding cells. The cells were stimulated at intervals of 10 days with irradiated peptide-pulsed autologous PBMC and 100 U/mL IL-2.

Adenosine 5′-triphosfate (ATP) Bioluminescent Assay

Adenosine 5′-triphosphate (ATP) Biolumescent Assay (CellTiter-Glo Luminescent cell viability Assay, Promega) allow to measure the quantity of ATP containing in the samples. ATP is consumed and light is emitted when firefly luciferase catalyzes the oxidation of D-luciferin.

Briefly, 100 μL of Assay Mix solution were added to a reaction vial for 3 minutes at room temperature; then rapidly were added 100 of sample diluent, mixed and immediately measured the amount of light produced. The final value is proportional to the amount of ATP in the sample.

### SN Preparation, Functional Assay and Mass Spectrometry

Supernatant (SN) from B16 or B16-OVA untreated or Salmonella treated cells were treated as follow. Proteins were removed from the SN through a Trichloroacetic acid (TCA)-based protein precipitation procedure. In detail, samples were incubated with TCA (13% final concentration) for 5 min at −20° C. and then for 5 hours at 4° C. Samples are then ultra-centrifuged at 15000 g for 15 min; peptides-enriched SN was collected and the pellet was discarded.

Products were loaded on the C18 matrix and washed with 0.1% Formic Acid. Finally, peptides are eluted with increasing percentages of Acetonitrile (5%, 10%, 20%, 50%, 80%).

Acetonitrile was removed by speed vacuum centrifuge.

Fractions were analyzed by matrix-assisted laser desorption/ionisation—time of flight mass spectrometry (MALDI-TOF MS). Full nLC-ESI spectrum [300-1650 Da] at 53.3 min was analyzed to confirm SIINFEKL (SEQ ID NO:1) identity.

The presence of functional SIINFEKL (SEQ ID NO:1) peptide, OVA(₂₅₇₋₂₆₄), was assessed by a functional assay using OT-I CD8 cells as describe in Materials and Methods.

### T2 Assay for Peptide Binding

The T2 binding assay is based upon the ability of peptides to stabilize the MHC class I complex on the surface of T2 cells.

T2 cells were incubated overnight at 37° C. at 2×105 cells/well in FCS-free RPMI medium with 100 μL of supernatant or MART-1 peptide (1 μM and 10 μM) as a positive control.

The next day, the cells were washed with FACS buffer (PBS, 0.1% sodium azide, 5% fetal bovin serum) and incubated for 10 min with blocking buffer (200 μg/mL mouse IgG in FACS buffer). Then, the cells were incubated for 20 minutes at +4° C. with BB7.2, an HLA-A2 conformation specific mouse antibody. The cells were washed twice with FACS buffer and fixed in paraformaldehyde for later acquisition by Accuri C6 Flow Cytometer (BD).

### Immunofluorescence Staining

OSA cells were washed twice with PBS and incubated for 30 minutes at room temperature with blocking buffer (PBS+0.03% of Tryton+2% FBS). Then, cells were incubated with Ab anti-phalloidin for 30 minutes at room temperature. The cells were washed twice with PBS and examined by fluorescent microscope.

### Alkaline Phosphatase Staining

OSA cells were washed twice with PBS and incubated with paraformaldeide (1%) for 10 minutes at room temperature. The cells were washed and incubated with NBT/BCIP solution for 1 hour at room temperature. Then, the cells were washed with PBS and examined microscopically.

In Vivo Immunization with Supernatant

Mice were immunized subcutaneusly with 100 μL of emulsion with supernatant containing released peptides and either Freund's Complete Adjuvant (CFA) or Freund's Incomplete Adjuvant (IFA). Nine days after the immunization, mice were killed, popliteal lymph nodes were smashed and cells were plated in flat-bottom 96-well plates and stimulated with PPD (3 μg/mL), OVA protein (30 μg/mL), OVA peptide 323-339 (3 μg/mL) or OVA peptide 257-264 (3 μg/mL). After 72 hours, the supernatant was collected and IFN-γ production was measured by ELISA.

To evaluate antibody response mice were killed four weeks after immunization, and serum was collected. The antibody titer to Salmonella and B16 antigens was evaluated by ELISA.

In Vivo Vaccination with DC Loaded Supernatant

DC1 dendritic cells, matured with LPS (1 μg/mL), were loaded with supernatant containing released peptides for 4 hours at 37° C. After incubation, the cells were washed twice and subcutaneously injected in the right flank of mice (on days 0 and 4). On 21 day 10⁵ B16 cells were subcutaneously injected in the left flank.

### Results

### Opening of Connexin Hemichannels by Salmonella Infection of Melanoma Cells

In the work of Saccheri et al (Saccheri, Pozzi et al. 2010) and in WO 2012/017033, it was shown that Salmonella induces, in melanoma cells, the up-regulation of connexin 43 (Cx43), a ubiquitous protein that forms gap junctions and that is often lost during carcinogenesis.

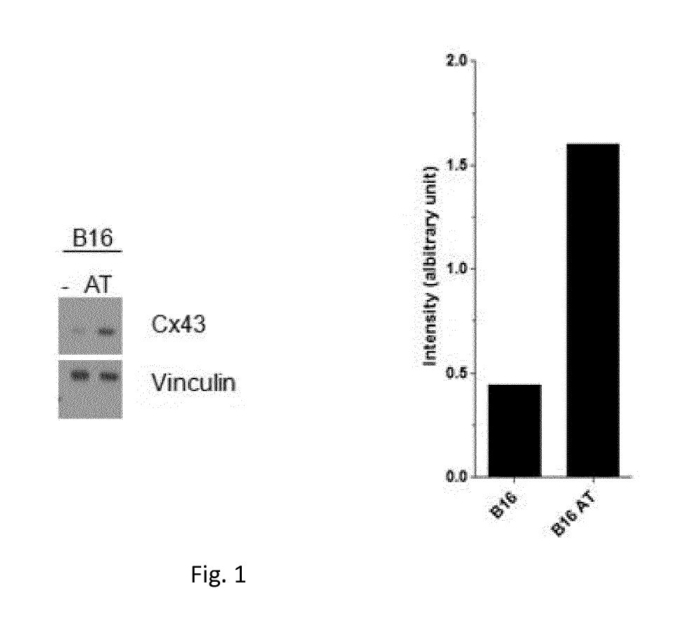

FIG. 1 confirmed that Cx43 expression was up-regulated in B16 melanoma cells after infection with the attenuated strain of Salmonella thyphimurium SL3261AT, as evaluated by Western blot analysis.

As described in the introduction, the single hemichannel that forms a gap junction in the plasma membrane is closed under resting conditions but can be induced to open under the influence of different stimuli (Saez, Retamal et al. 2005).

In order to assess whether Salmonella is able to stimulate the opening of connexin hemichannels on the surface of melanoma cells, inventors measured ATP extracellular concentration using an adenosine 5′-thriphosfate (ATP) bioluminescent assay (as described in Materials and Methods) in Salmonella treated B16 cells.

Briefly, inventors used the attenuated strain of Salmonella thyphimurium SL3261AT to infect mouse melanoma cell line B16 and the same cells expressing ovalbumin (B16 OVA), previously treated or not with the gap-junction blocker, heptanol.

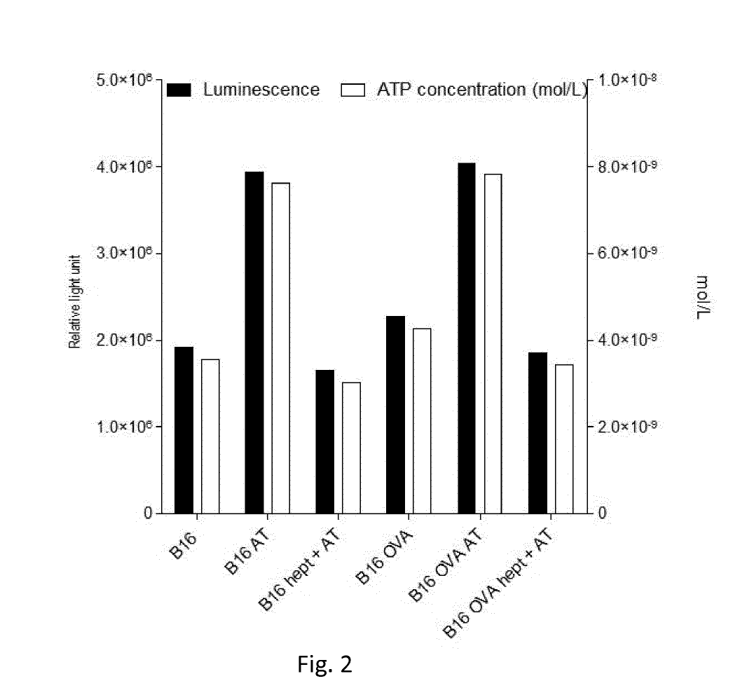

As shown in FIG. 2, Salmonella infection induces the release of extracellular ATP from both cell lines and this effect is significantly reduced by the gap junction blocker heptanol. This result indicates that a cytoplasmatic molecule can be released by connexin hemichannel (CxH) in a Salmonella dependent manner, demonstrating the role of bacteria to stimulate the opening of hemichannels, in a condition where it induces the up-regulation of Cx43.

### Identification of Released Endogenous Peptides Functional Assay and Mass Spectrometry

SIINFEKL (SEQ ID NO:1) H-2Kb restricted OVA octapeptide (OVA ₂₅₇₋₂₆₄) is known to be processed and presented by B16-OVA MHC class I molecules. To identify this prototype endogenous peptide, released in the supernatant, inventors used the attenuated strain of Salmonella thyphimurium SL3261AT (SL) to infect mouse melanoma cell line B16 and the same cells expressing ovalbumin (B16 OVA). B16 OVA derived SN treated as described in material and methods were analyzed both functionally and biochemically to detect the presence of (OVA ₂₅₇₋₂₆₄) in the derived fractions.

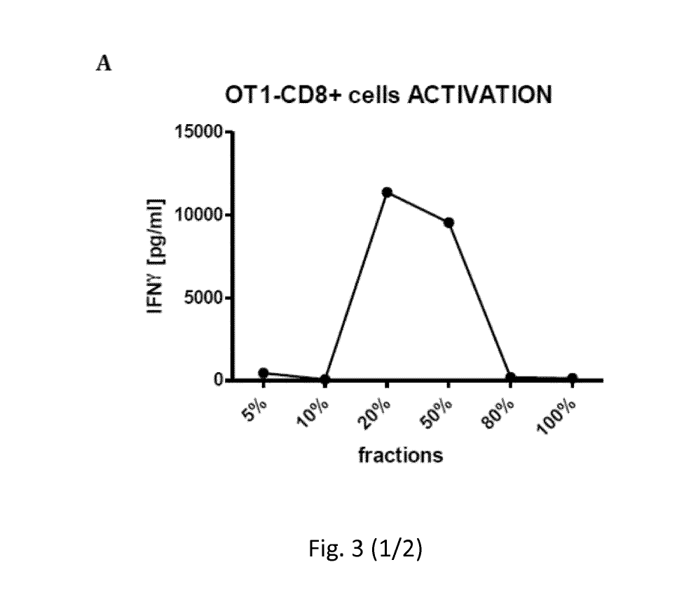

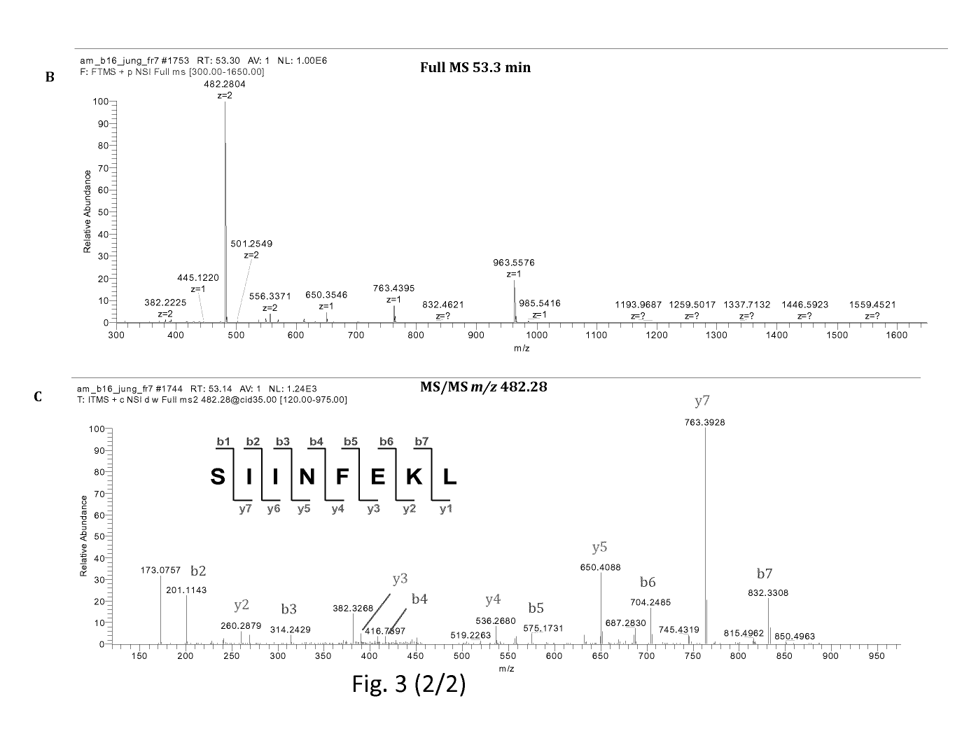

As shown in FIG. 3a CD8 OT-I T cells activation measured, as IFN-gamma released, is mainly present in SN fractions 20% and 50%, which were further analyzed by mass spectrometry.

Full MS, FIG. 3b, and full nLC-ESI, FIG. 3c, spectra [300-1650 Da] at 53.3 min of B16 OVA-derived SN was performed and analysis of nLC-ESI spectrogram confirm the SIINFEKL (SEQ ID NO:1) identity demonstrating the release of a processed endogenous peptide in SN.

### Gap-Junction Hemichannel Dependent Tumor Peptides Release by Salmonella-Infected Tumor Cells

Inventors continued their investigation asking whether cytoplasmic peptides could be transferred in a CxH dependent manner by Salmonella infected tumor cells to the extracellular milieu.

In order to investigate the release of MHC class I peptides by Salmonella infected tumor cells, inventors exploited the ability of exogenous peptides to stabilize the MHC class I complex on the surface of T2 cells. T2 are an HLA-A0201 hybrid human cell line lacking TAP-2 (transporter-associated with antigen processing) and consequently are defective in loading class I molecules with antigenic peptides generated in the cytosol. This leads to very unstable MHC class I molecules on the cell surface. The association of exogenously added peptides stabilizes surface expression of HLA molecules, recognizable by specific anti-HLA-A0201 antibody.

Briefly, inventors infected human melanoma cells SKmel-24 and colorectal adenocarcinoma cells HT29, with Vivofit®, an oral typhoid vaccine that contains live, attenuated cells of the bacteria Salmonella enterica serovar Thyphi (TY21a), and inventors collected the supernatant as described in Materials and Methods. T2 cells were incubated with the supernatant obtained from uninfected or infected cells treated or not with the gap junction blocker (heptanol). Surface expression of HLA-A0201 was evaluated using a conformation-specific mouse antibody, as described in Materials and Methods.

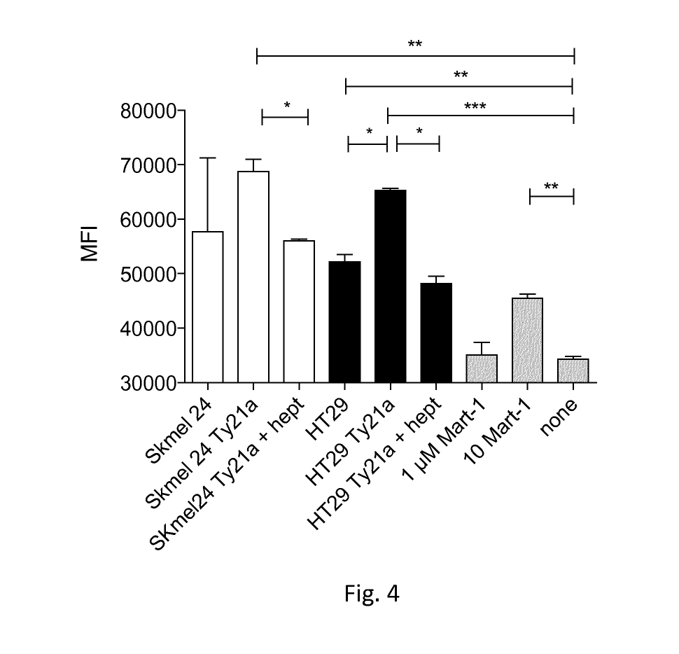

FIG. 4 shows that T2 cells incubated with Mart-1 (26-35) peptide, as positive control, display a level of HLA-A0201 mean fluorescence intensity (MFI) significantly higher than that of unloaded T2 cells (none), indicating that the presence of the exogenous peptides is capable of stabilizing MHC class I complexes on the surface of T2 cells. Incubation of T2 cells with supernatant obtained from infected tumor cells increases the MFI that is abolished by the gap junction blocker. These results demonstrate that peptides from Salmonella infected tumor cells are released in a CxH-dependent manner and they can bind to MHC class I molecules.

Similar results were obtained using supernatants collected from another tumor cell line (colorectal adenocarcinoma cells) suggesting that this phenomenon could be widely applied to other type of cancer cells.

Since infected tumor cells are able to release peptides in a CxH-dependent manner, inventors tested whether antigen-specific T cells could recognize those peptides. Murine dendritic cells (D1), previously incubated with the supernatant obtained from B16 cells expressing ovalbumin (B16-OVA) infected or not with Salmonella SL3261AT, were cocultured with the OVA specific-B3Z hybridoma T cells. After 72 hours, the amount of IL-2 secretion was assessed by ELISA as a measure of OVA peptide recognition.

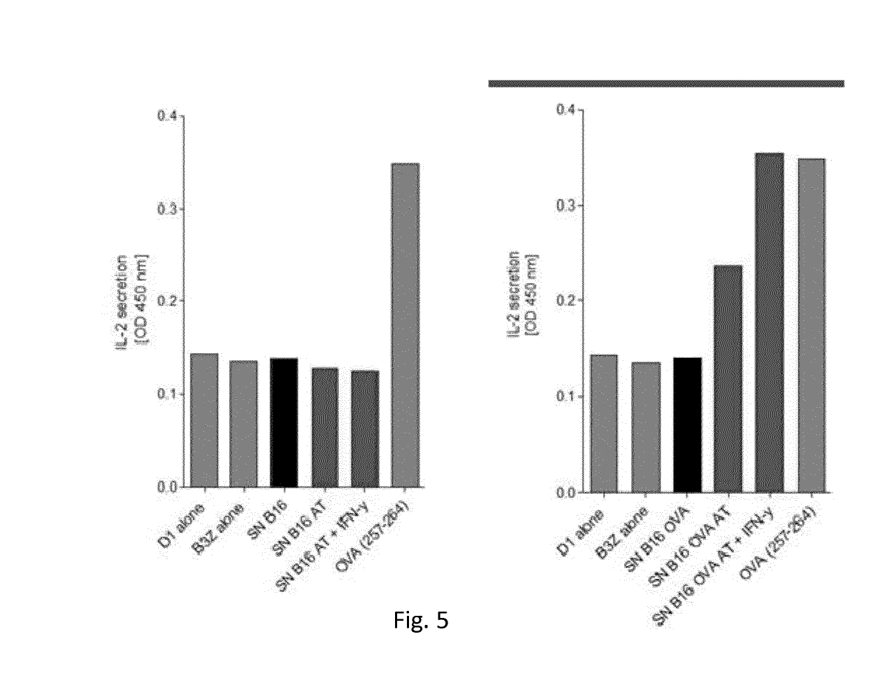

In FIG. 5 it is shown that dendritic cells incubated with supernatant obtained from B16-OVA cells treated with Salmonella alone or in combination with IFN-γ activate OVA-specific T cells as shown by the increase of IL-2 secretion. This result demonstrates that the antigenic peptides released by Salmonella infected tumor cells are recognized and are able to activate antigen-specific T cells.

By contrast, when dendritic cells loaded with supernatant obtained from B16 cells, were co-cultured with OVA specific T cells there was no production of IL-2, demonstrating the absence of OVA peptide and the specificity of the assay.

Based on this evidence, inventors decided to investigate the release of tumor peptides by Salmonella infected human tumor cell lines.

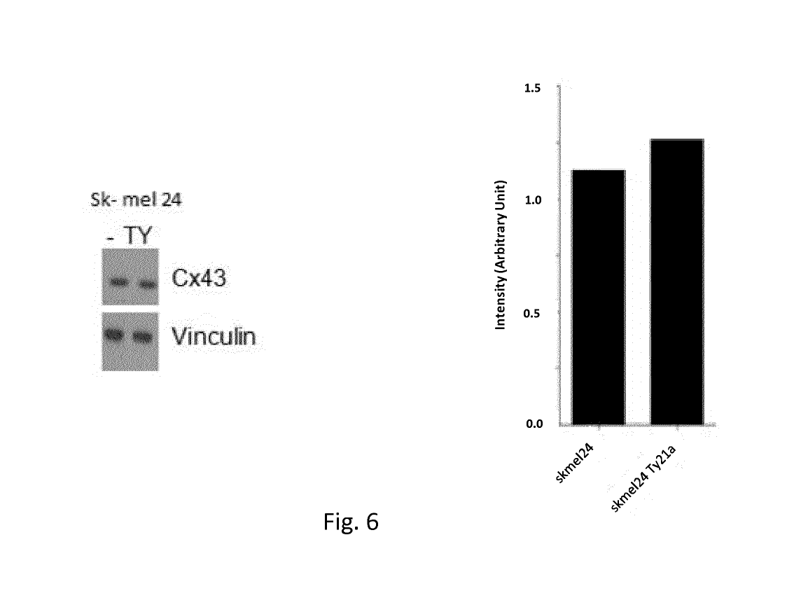

Inventors analysed, by Western blot analysis, the effect of Salmonella infection on Cx43 expression in human melanoma cells, SK-mel 24. As shown in the FIG. 6, Cx43 expression is high already in resting conditions and after Salmonella infection is slightly up-regulated.

To evaluate the release of antigenic peptides inventors infected human melanoma cells SKmel24 and 624.38 with Salmonella TY21a in presence or not of heptanol and the supernatant was harvested as described in Materials and Methods.

Human monocyte derived DCs differentiated in vitro from monocytes with GM-CSF and IL-4 (moDCs) were incubated with the supernatant, produced as indicated above, and cocultured with Mart-1 (26-35) specific CTLs (obtained as described in Materials and Methods). After 72 hours of coculture, the amount of IFN-gamma secretion was measured by ELISA.

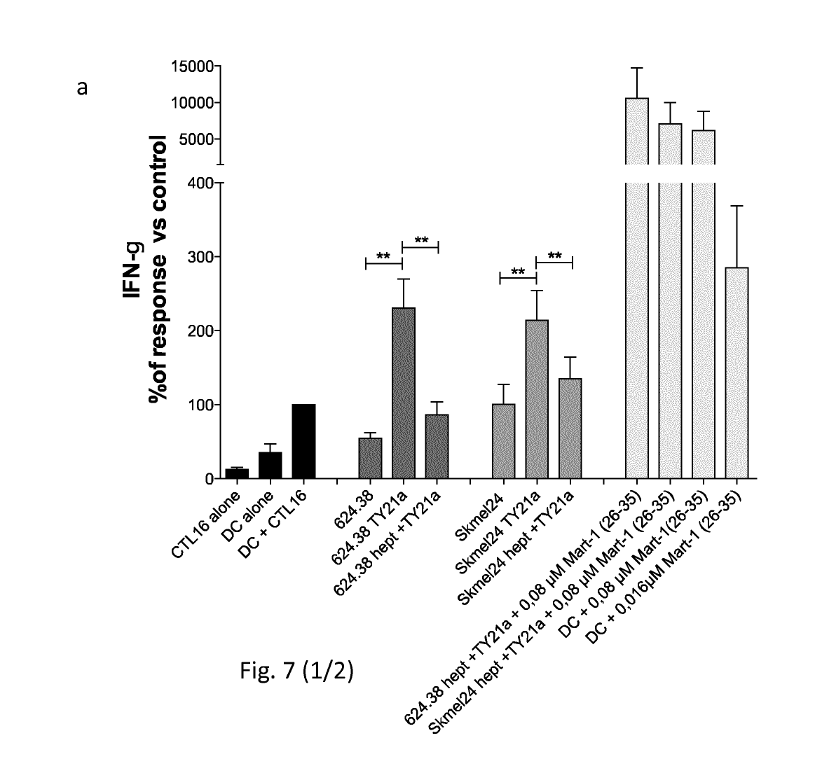

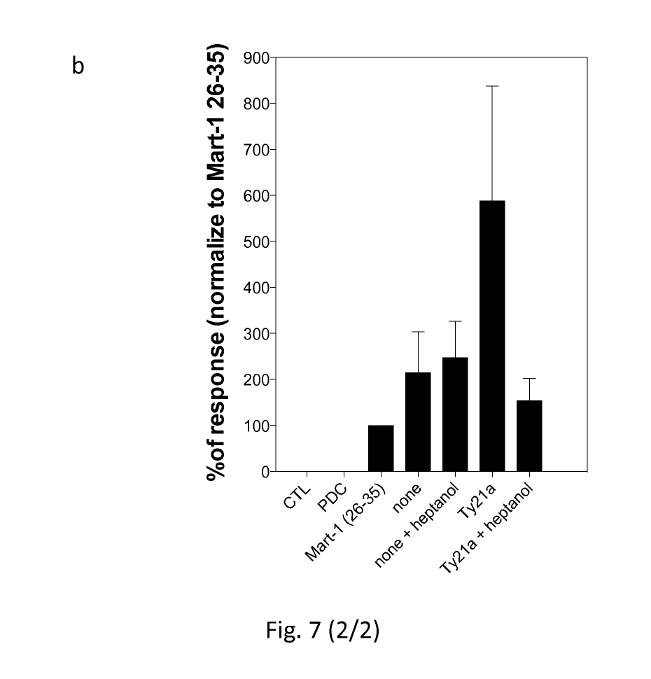

FIG. 7a and FIG. 7b show respectively that moDCs or PDC incubated with supernatant of melanoma infected tumor cells, activate Mart-1 (26-35) specific human CTLs and the gap junction blocker significantly inhibits this effect. Exogenous addition of Mart-1 (26-35) peptide restored completely the response.

This result demonstrates a CxH-dependent release of tumor peptides by Salmonella infected human melanoma cell lines and their ability to activate tumor antigen-specific human CTLs.

### Release of Tumor Peptides by Salmonella Infected Tumor Cells Induces an In Vivo Antitumor Immune Response

To investigate whether the tumor peptides released from infected tumor cells could induce an in vivo immune response, C57BL/6J mice were immunized in the footpad with supernatant, obtained from B16 OVA cells infected or not with Salmonella SL3261AT, emulsified with Freund's complete adjuvant (CFA).

Briefly, nine days after immunization, the popliteal lymph nodes were removed and cells were stimulated in vitro with PPD (Tuberculin purified protein derivated), as a positive control, and OVA protein. After 72 hours of incubation, the amount of IFN-gamma secretion was assessed by ELISA to evaluate the activation of immune cells.

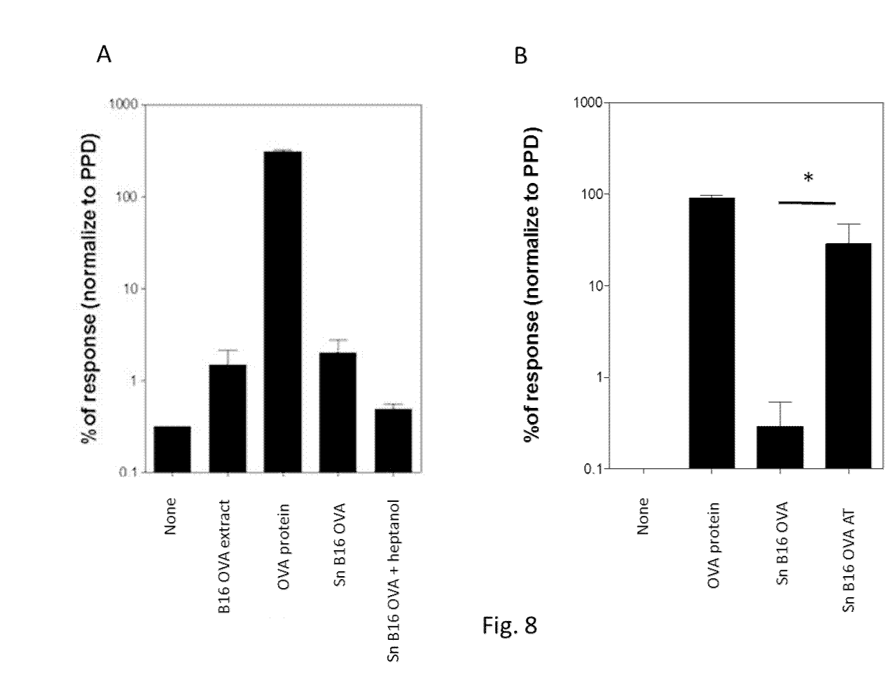

As shown in FIG. 8A, immunization with the supernatant obtained from B16 OVA cells induced IFN-gamma production after in vitro recall with OVA protein that was reduced by heptanol. This response is similar to that induced after immunization with B16 OVA extract. Instead, as shown in FIG. 8B, when the mice were immunized with the supernatant obtained from Salmonella infected B16 OVA (SN B16 OVA AT), the IFN-gamma secretion was increased. These results demonstrate that peptides released by Salmonella infected tumor cells can induce an in vivo immune response and such response is CxH-dependent.

In order to identify the type of released tumor antigens, C57BL/6J mice were immunized subcutaneously with supernatant obtained from Salmonella infected or uninfected B16 cells treated or not with the gap-junction blocker (heptanol) or the proteasome inhibitor (MG132), emulsified with Freund's Incomplete adjuvant (IFA). Four weeks later inventors evaluated the amount of total IgG specific to Salmonella SL3261AT and to mouse melanoma cell line B16 by ELISA.

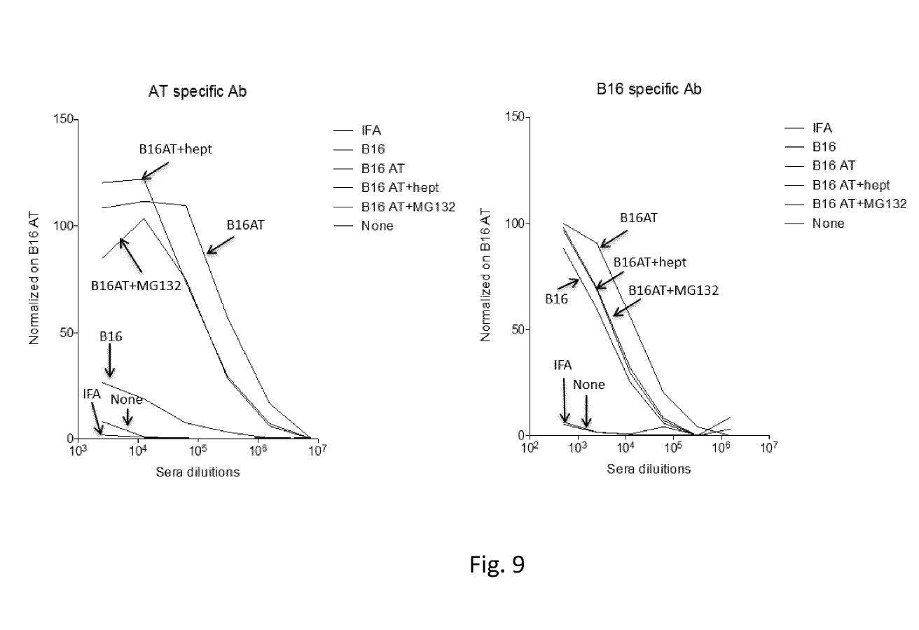

FIG. 9A shows that immune sera from mice immunized with the supernatant obtained from infected B16 cells recognize Salmonella antigens and this response is reduced by heptanol and MG132. This data suggests that Salmonella after infection is processed and peptides are released in the supernatant through CxH, as shown by the effect of heptanol. Importantly, these peptides are partly generated by proteasome processing because the antibody response is reduced by MG132 treatment.

FIG. 9B shows that Salmonella infection increased the release of B16 proteasome processed tumor peptides as indicated by the presence of B16 specific antibody in the serum of mice immunized with supernatant obtained from infected B16 cells.

This effect is reduced by heptanol and MG132, underlining the release of proteasome-processed tumor peptides by CxH.

Moreover, in FIG. 9B, it was shown that an antibody response against tumor peptides was induced also in mice immunized with supernatant obtained from uninfected B16 cells. However, the titer of antibody response is significantly lower than that of Salmonella infection's group, probably due to the lower concentration of tumor peptides release in the supernatant.

These results indicate the induction of an in vivo immune response by pre-processed tumor peptides released by Salmonella infected tumor cells.

In order to investigate the effect on tumor growth induced by the released pre-processed tumor peptides, inventors decided to use dendritic cells as adjuvant.

Briefly, inventors vaccinated C57BL/6J mice with murine dendritic cells (D1) previously loaded with supernatant obtained from non infected or infected B16 cells, twice (on days 0 and 4) before the challenge with B16 cells (day 21).

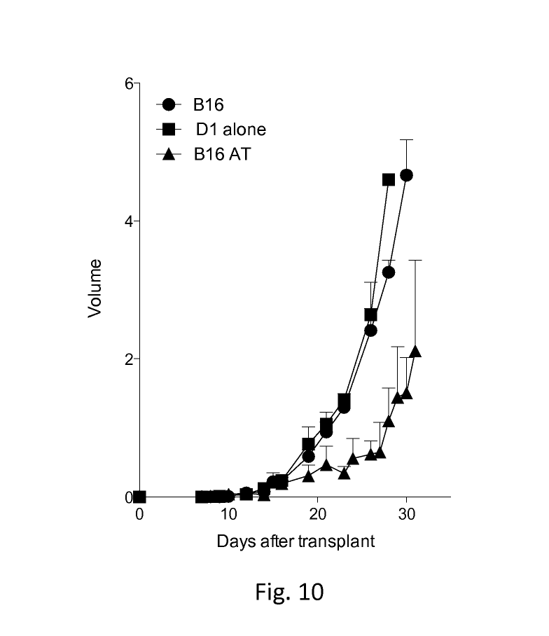

In FIG. 10, it is shown a statistically significant delay of tumor growth in mice vaccinated with DCs loaded with supernatant obtained from Salmonella infected B16 cells (red line) and the effect was lost when DCs were loaded with supernatant obtained from uninfected B16 cells (black line). The tumor peptides released by Salmonella infected tumor cells were captured and cross-presented by dendritic cells inducing an in vivo antitumor immune response.

These data confirm that tumor peptides released by Salmonella infected tumor cells are able to induce an in vivo anti-tumor immune response.

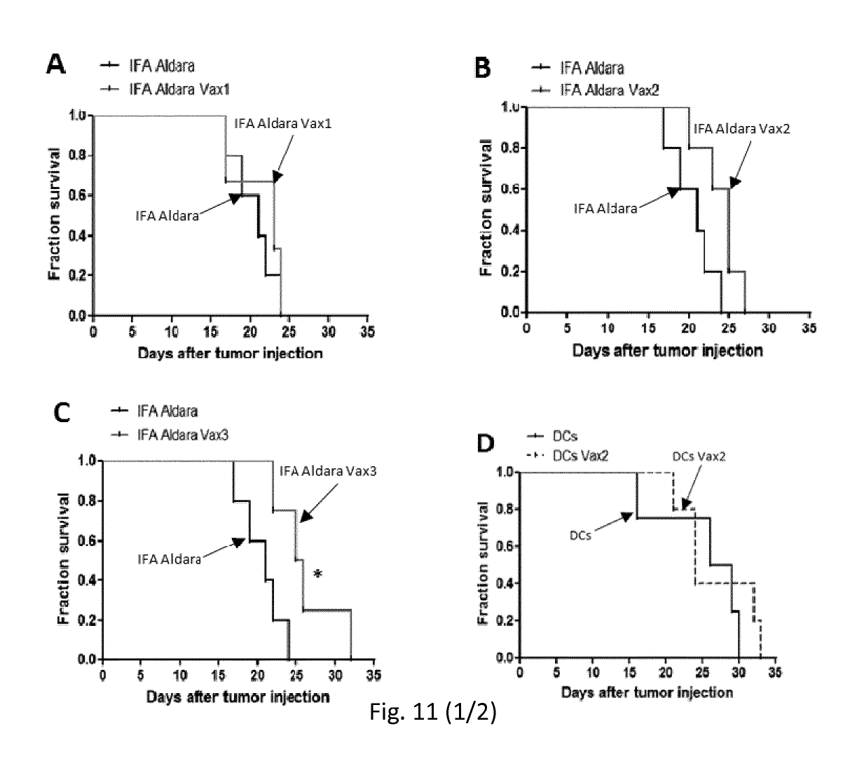

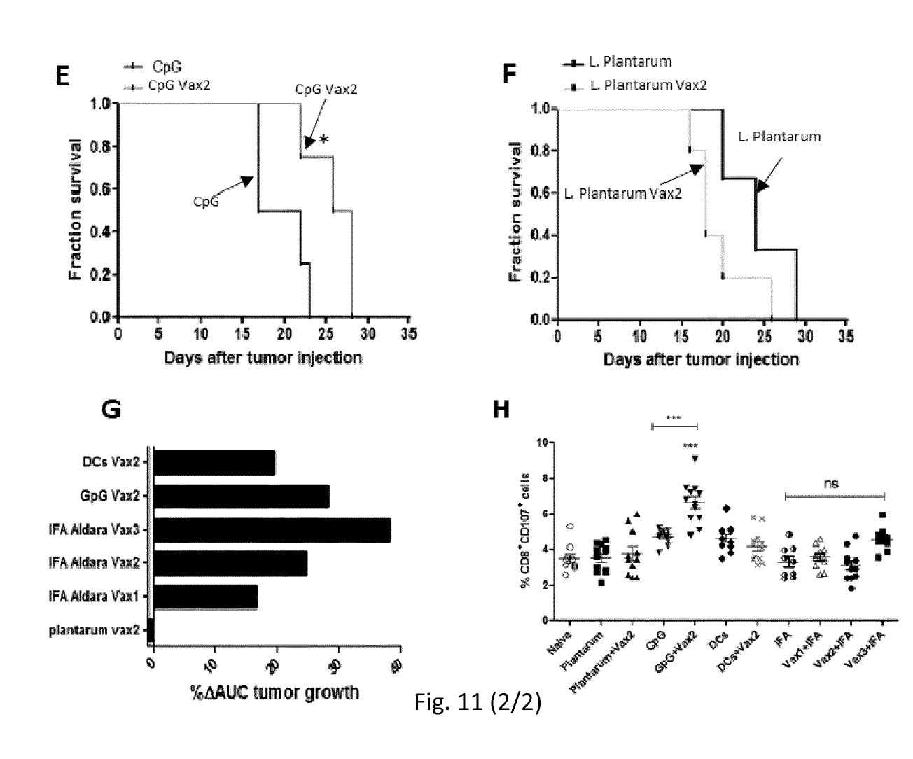

The inventors also assessed the effect of a three increasing doses of supernatant not in association with dendritic cells, to evaluate whether they could induce an immune response without exogenous dendritic cells but targeting endogenous cells so to use the peptides as a vaccine. They combined the peptides with different adjuvants: Incomplete Freund's adjuvant (IFA) plus Aldara (Imiquimod: a Toll-like receptor (TLR)-7 agonist, or ODN1826 (CpG Vax). Mice vaccinated with an increasing dose of supernatant equivalent to 0.5×10⁶ cells (Vax1), 2×10⁶ cells (Vax2), 5×10⁶ cells (Vax3) showed a positive correlation between dose and antitumor response in terms of overall survival (FIG. 11A-C). Mice vaccinated with CpG-Vax had a high cytotoxic T lymphocyte degranulation in their peripheral blood mononucleated cells (PBMCs); a similar trend was observed in PBMCs isolated from mice immunized with the higher dose of peptides-based vaccine combined with IFA and Aldara (IFA Aldara Vax3) (FIG. 11 H). Consistently, they observed that both CpG Vax and IFA Aldara Vax3 mice had a significant prolonged survival compared with their control group (respectively CpG and IFA Aldara, FIGS. 11 C and E). Interestingly, vaccination mediated by dendritic cells loaded with peptides (DC Vax2) did not induce any increased antitumor response (FIG. 11D) suggesting that targeting endogenous DCs may be even more efficient. The inventors also tested the adjuvant role of a L. plantarum administered to mice before the immunization procedure (FIG. 11F). Unexpectedly, the oral administration of L. plantarum did not augment the effect of the immunization.

### Mass Spectrometry Analysis Identify Pool of Endogenous Peptides Differentially Released by B16 Salmonella Infected Cells

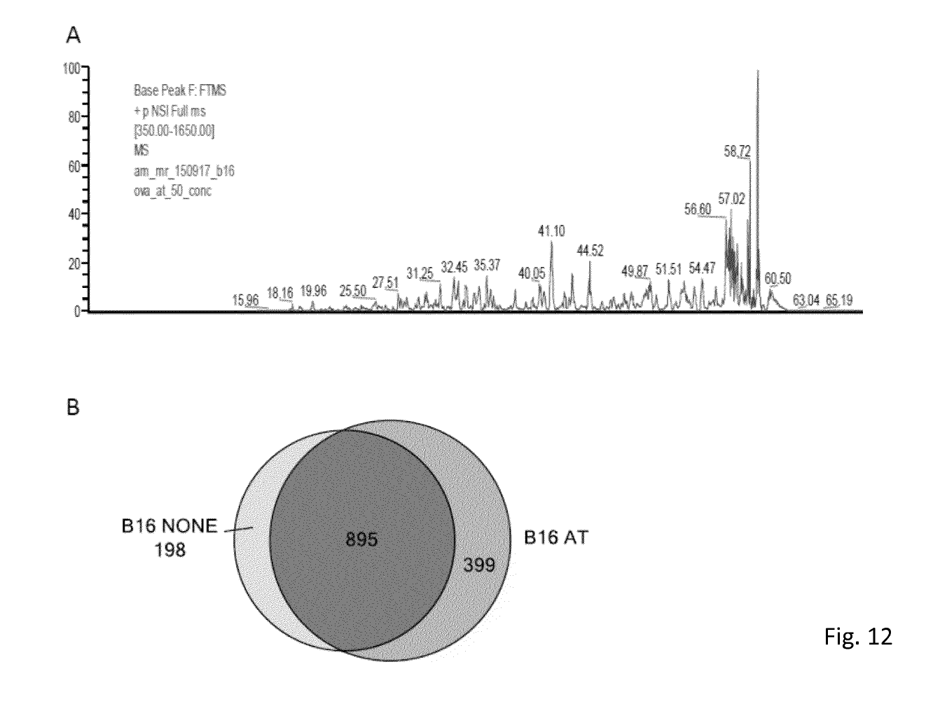

In order to assess whether endogenous peptides are differentially released in the SN by SL infection, B16 cell were infected with SL and SN recovered and treated as describe in material and methods. FIG. 12a showed full MS spectra of B16 infected SN, and Venn diagram FIG. 12b highlight 399 protein associated to the detected and sequence peptides, that are differentially released in the SN of B16 SL-infected cells.

These data indicate that the ability to induce an in vivo anti-tumor immune response reside in a pool of immunogenic tumor peptides released by Salmonella infected tumor cells.

The inventors assessed the nature of the differentially produced peptides.

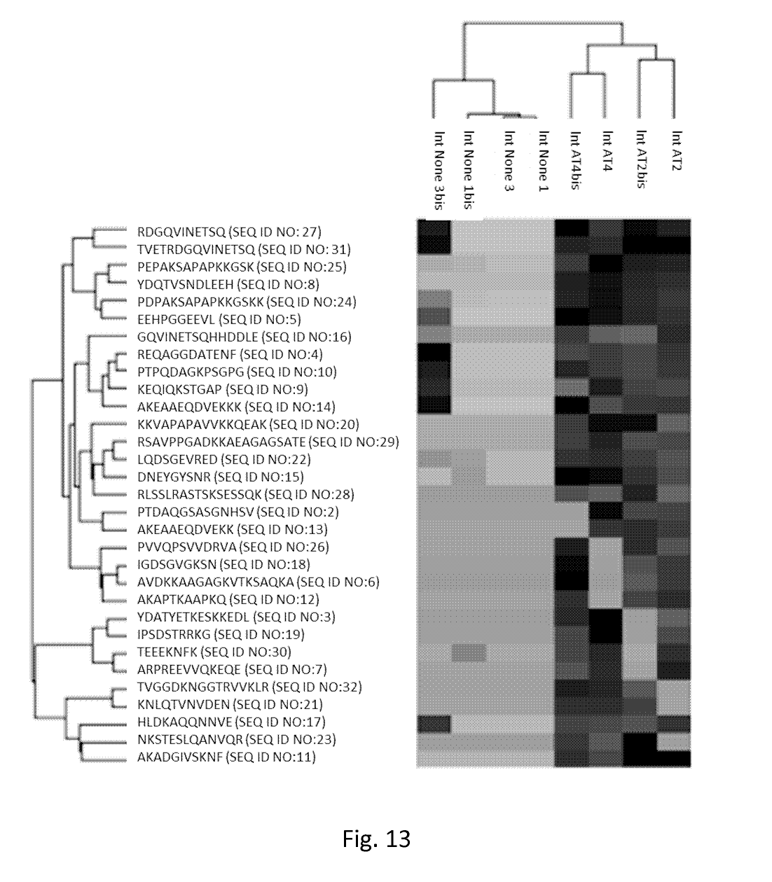

Owing to MaxQuant software that quantitatively analyze the sequenced and identified peptides they found that 31 peptides were significantly more abundant inside Vax samples (FIG. 13) of which 9 were selected based on their capacity to bind the MHC class I molecule (Table I). Importantly one of the statistically-selected Vax peptides is a known tumor antigen and nine peptides were predicted to be good MHC binders by Immuno Epitopes Data Base (IEDB) (SEQ ID Nos:2-10), suggesting that they could be potential novel epitopes (Table I).

### Translational Study of Canine Osteosarcoma

Starting from these preliminary results, inventors then investigated whether this strategy could be translatable to other types of tumors, testing our approach in an experimental veterinary study for the treatment of a deadly form of spontaneous canine osteosarcoma.

Tumor specimens were obtained from a Veterinary clinic and primary osteosarcoma cell lines were generated as described in Materials and Methods.

Experimental vaccine containing the supernatant collected after infection of canine osteosarcoma cells with the vaccine strain Vivofit® of Salmonella enterica serovar thyphi (TY21a) (as described in Materials and Methods) was produced for all dog patients.

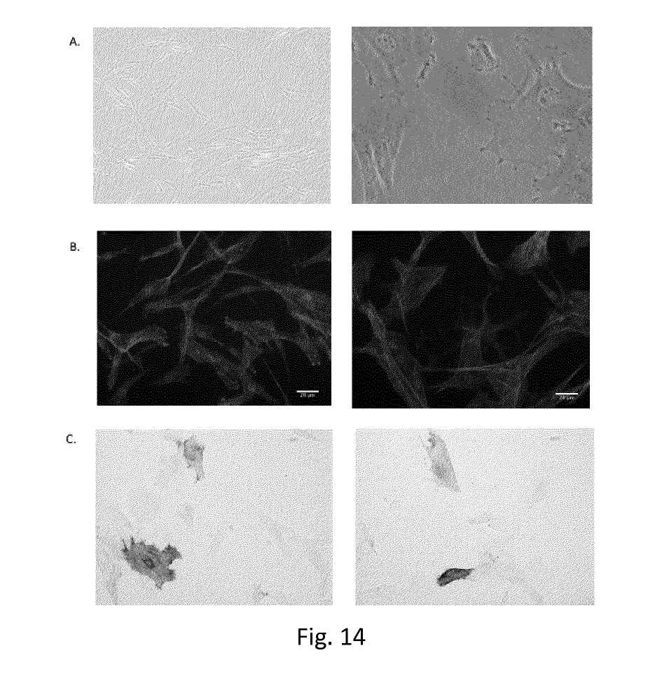

In order to induce the shrinkage of the tumor and afterwards a long lasting anti-tumor immune response, the treatment schedule includes the association of standard chemotherapy (4 cycles of carboplatin every 21 days) with experimental vaccination. Vaccination started after 2 cycle of standard chemotherapy. Two cycle of vaccination were administered intradermically, after topical application of 5% Imiquimod cream (Aldara®) on the injection site with the following modality: first cycle of 2 vaccinations at 21 days intervals and second cycle of 4 vaccinations at 30 days intervals. The patients will remain under observation for the following 24 h in the veterinary clinic. FIG. 14 shows primary canine osteosarcoma cells obtained from dog's osteosarcoma specimen. Panel A shows OSA cells analyzed by optical microscope: the cells appear adherent, mostly elongated of varying size or large pentagonal and polyhedral. There are numerous characteristic cytoplasmatic granules and vacuoles in most cells.

Panel B shows OSA cells stained with anti-phalloidin antibody to visualize actin filaments and with DAPI for nuclear counterstain. Moreover, inventors identified OSA cells by staining them for alkaline phosphatase activity (panel C).

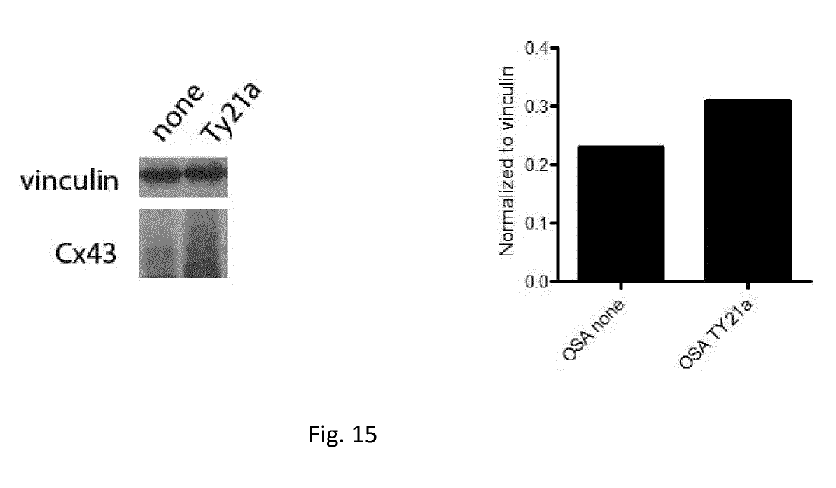

Finally, inventors characterized the effect of Salmonella on connexin 43 (Cx43) in canine osteosarcoma cells. As evaluated by Western blot analysis (FIG. 15), Salmonella Ty21a is able to up-regulate Cx43 expression in osteosarcoma cells.

Inventors enrolled twenty osteosarcoma or high grade sarcoma dog patients and eight of these started the experimental therapy.

Prognosis for dogs suffering from osteosarcoma, undergoing surgery and chemotherapy is approximately 235-360 days after diagnosis. Since from one dog inventors could not obtain the specimen to generate the cell line, inventors decided to treat it using the vaccine generated from another dog in heterologous fashion.

Present results (Table II) indicate that three osteosarcoma patients died before completing the vaccination (OSA 1, 8 and 23), two are under vaccination (OSA 25 and OSA 29) and one patient (OSA 0) had a long overall survival, dying after 653 days for reasons not related to the tumor. With regard to the sarcoma affected dogs (SA), one patient is alive but still within the life expectancy period (SA 19), and the second patient is alive after more than 1073 days and survived also to a recurrence of disease (SA 5) that was again treated with vaccination, showing that a marked antitumor response has been promoted.

TABLE I
List of the Vax-specific peptides selected for their MHC-binding capability (predicted
by IEDB in silico tool as percentile rank).

| | | MHC binding |
|---|---|---|
| Sequence | Protein names | prediction |
| (SEQ ID NO: 2) PTDAQGSASGNHSV | Poimin | 0.7 |
| (SEQ ID NO: 3) YDATYETKESKKEDL | Cofilin | 0.8 |
| (SEQ ID NO: 4) REQAGGDATENF | Cytochrome b5 | 0.8 |
| (SEQ ID NO: 5) EEHPGGEEVL | Cytochrome b5 | 0.9 |
| (SEQ ID NO: 6) AVDKKAAGAGKVTKSAQKA | Elongation factor 1-alpha | 1.5 |
| (SEQ ID NO: 7) ARPREEVVQKEQE | Eukaryotic translation initiation factor 4H | 1.6 |
| (SEQ ID NO: 8) YDQTVSNDLEEH | Ras GTPase-activating protein-binding protein 1 | 1.8 |
| (SEQ ID NO: 9) KEQIQKSTGAP | Cleavage stimulation factor subunit 2 | 2.1 |
| (SEQ ID NO: 10) PTPQDAGKPSGPG | A disintegrin and metalloproteinase with | 2.2 |
| | thrombospondin motifs 1 | |

TABLE II
Treatment schedule

| | | | | | | | | Survival |
|---|---|---|---|---|---|---|---|---|
| Patient | | Breed | Diagnosis | | VAX | | | (from diagnosis) |
| OSA0 | Penelope | Terranova | osteosarcoma | Dec. 15, 2012 | Heterologous | Mar. 11, 2013 | Completed | dead after 653 |
| OSA1 | Kira | Amstaff | osteosarcoma | Jan. 18, 2013 | Autologous | Apr. 3, 2013 | 5 out of 6 | dead after 224 |
| OSA8 | Zorro | Meticcio | osteosarcoma | Dec. 1, 2013 | Autologous | Mar. 27, 2014 | 5 out of 6 | dead after 229 days |
| OSA23 | Lacy | Rottweiler | osteosarcoma | Nov. 24, 2015 | Autologous | | 5 out of 6 | dead after 273 days |
| OSA 25 | Balti | Rottweiler | osteosarcoma | Feb. 16, 2016 | Autologous | | on going | 236 |
| OSA29 | Margot | Tosa Inu | osteosarcoma | | Autologous | | On going | |
| SA 5 | Luna | Pitbull | sarcoma | November 2013 | Autologous | Feb. 17, 2014 | Completed | alive after 1073 days |
| | | | sarcoma relapse | November 2015 | Autologous | Feb. 20, 2016 | Completed | |
| SA 19 | Shary | Rottweiler | sarcoma | Aug. 25, 2015 | Autologous | Oct. 20, 2015 | completed | 411 |

TABLE III
List of the peptides identified by Mass spectrometry as differentially present in the
supernatant of bacteria-treated versus untreated melanoma cells in FIG. 13

| | | Gene | |
|---|---|---|---|
| Sequence | Proteins | name | Protein name |
| AKADGIVSKNF (SEQ ID NO: 11) | Q9CQR2 | Rps21 | 40S ribosomal protein S21 |
| AKAPTKAAPKQ (SEQ ID NO: 12) | Q8BP67 | Rpl24 | 60S ribosomal protein L24 |
| AKEAAEQDVEKK (SEQ ID | P47963 | Rpl13 | 60S ribosomal protein L13 |
| NO: 13) | | | |
| AKEAAEQDVEKKK (SEQ ID | P47963 | Rpl13 | 60S ribosomal protein L13 |
| NO: 14) | | | |
| ARPREEVVQKEQE (SEQ ID NO: 7) | Q9WUK2 | Eif4h | Eukaryotic translation initiation factor 4H |
| AVDKKAAGAGKVTKSAQKA (SEQ | Q58E64 | Eef1a1 | Elongation factor 1-alpha |
| ID NO: 6) | | | |
| DNEYGYSNR (SEQ ID NO: 15) | S4R1W1 | Gm3839 | Glyceraldehyde-3-phosphate dehydrogenase |
| EEHPGGEEVL (SEQ ID NO: 5) | G5E850 | Cyb5a | Cytochrome b5 |
| GQVINETSQHHDDLE (SEQ ID | Q5FWJ3 | Vim | Vimentin |
| NO: 16) | | | |
| HLDKAQQNNVE (SEQ ID NO: 17) | F6SVV1 | Gm9493 | 40S ribosomal protein S7 |
| IGDSGVGKSN (SEQ ID NO: 18) | G3UY29 | Rab11b | Ras-related protein Rab-11A |
| IPSDSTRRKG (SEQ ID NO: 19) | P62264 | Rps14 | 40S ribosomal protein S14 |
| KEQIQKSTGAP (SEQ ID NO: 9) | A2AEK1 | Cstf2 | Cleavage stimulation factor subunit 2 |
| KKVAPAPAVVKKQEAK (SEQ ID | | | |
| NO: 20) | Q58ET1 | Rpl7a | 60S ribosomal protein L7a |
| KNLQTVNVDEN (SEQ ID NO: 21) | Q5M9K9 | Rpl31 | 60S ribosomal protein L31 |
| LQDSGEVRED (SEQ ID NO: 22) | J3QPS8 | Eif5a | Eukaryotic translation initiation factor 5A-1 |
| NKSTESLQANVQR (SEQ ID | P47963 | Rpl13 | 60S ribosomal protein L13 |
| NO: 23) | | | |
| PDPAKSAPAPKKGSKK (SEQ ID | Q64525 | Hist2h2bb | Histone H2B type 2-B |
| NO: 24) | | | |
| PEPAKSAPAPKKGSK (SEQ ID | Q6ZWY9 | Hist1h2bc | Histone H2B type 1-C/E/G |
| NO: 25) | | | |
| PTDAQGSASGNHSV (SEQ ID | D6REH0 | Tmem123 | Porimin |
| NO: 2) | | | |
| PTPQDAGKPSGPG (SEQ ID | P97857 | Adamts1 | A disintegrin and metalloproteinase with |
| NO: 10) | | | thrombospondin motifs 1 |
| PVVQPSVVDRVA (SEQ ID | Q9DBG5 | Plin3 | Perilipin-3 |
| NO: 26) | | | |
| RDGQVINETSQ (SEQ ID NO: 27) | Q5FWJ3 | Vim | Vimentin |
| REQAGGDATENF (SEQ ID NO: 4) | G5E850 | Cyb5a | Cytochrome b5 |
| RLSSLRASTSKSESSQK (SEQ ID | Q5BLK1 | Rps6 | 40S ribosomal protein S6 |
| NO: 28) | | | |
| RSAVPPGADKKAEAGAGSATE | Q5M9K7 | Rps10 | 40S ribosomal protein S10 |
| (SEQ ID NO: 29) | | | |
| TEEEKNFK (SEQ ID NO: 30) | P47963 | Rpl13 | 60S ribosomal protein L13 |
| TVETRDGQVINETSQ (SEQ ID | Q5FWJ3 | Vim | Vimentin |
| NO: 31) | | | |
| TVGGDKNGGTRVVKLR (SEQ ID | Q3UCH0 | Rpl6 | 60S ribosomal protein L6 |
| NO: 32) | | | |
| YDATYETKESKKEDL (SEQ ID | F8WGL3 | Cfl1 | Cofilin-1 |
| NO: 3) | | | |
| YDQTVSNDLEEH (SEQ ID NO: 8) | P97855 | G3bp1 | Ras GTPase-activating protein-binding protein |
| | | | 1 |

### REFERENCES

Mocellin, S. (2012). “Peptides in melanoma therapy.” Current pharmaceutical design 18(6): 820-831.
Adams, S., M. A. Lowes, et al. (2008). “Lack of functionally active Melan-A(26-35)-specific T cells in the blood of HLA-A2+ vitiligo patients.” The Journal of investigative dermatology 128(8): 1977-1980.
Fourcade, J., Z. Sun, et al. (2010). “Human tumor antigen-specific helper and regulatory T cells share common epitope specificity but exhibit distinct T cell repertoire.” Journal of immunology 184(12): 6709-6718.
Speiser, D. E., P. Baumgaertner, et al. (2008). “Unmodified self antigen triggers human CD8 T cells with stronger tumor reactivity than altered antigen.” Proceedings of the National Academy of Sciences of the United States of America 105(10): 3849-3854.
Meijer, S. L., A. Dols, et al. (2007). “Induction of circulating tumor-reactive CD8+ T cells after vaccination of melanoma patients with the gp100 209-2M peptide.” Journal of immunotherapy 30(5): 533-543.
Knittelfelder, R., A. B. Riemer, et al. (2009). “Mimotope vaccination—from allergy to cancer.” Expert opinion on biological therapy 9(4): 493-506
van Stipdonk, M. J., D. Badia-Martinez, et al. (2009). “Design of agonistic altered peptides for the robust induction of CTL directed towards H-2Db in complex with the melanoma-associated epitope gp100.” Cancer research 69(19): 7784-7792.
Parmiani, G., C. Castelli, et al. (2002). “Cancer immunotherapy with peptide-based vaccines: what have we achieved? Where are we going?” Journal of the National Cancer Institute 94(11): 805-818.
Saccheri, F., C. Pozzi, et al. (2010). “Bacteria-induced gap junctions in tumors favor antigen cross-presentation and antitumor immunity.” Science translational medicine 2(44): 44ra57.
Saez, J. C., M. A. Retamal, et al. (2005). “Connexin-based gap junction hemichannels: gating mechanisms.” Biochimica et biophysica acta 1711(2): 215-224.

1. A method for obtaining a cell culture supernatant or a fraction thereof comprising a specific tumor antigen peptide repertoire comprising the steps of:
a) exposing in suitable conditions a tumor cell culture to a Pattern Recognition Receptor (PRR) agonist and/or to one inflammatory cytokine to increase the opening of connexin-hemichannels (CxH);
b) collecting the cell culture supernatant; and
c) optionally obtaining a fraction of said supernatant,
wherein said supernatant comprises MHC class I and MHC class II peptides and non-classical MHC molecules (HLA-E).

2. The method for obtaining a specific tumor antigen peptide repertoire loaded and/or activated dendritic cell comprising the steps of:
a) exposing in suitable conditions a tumor cell culture to a Pattern Recognition Receptor (PRR) agonist and/or to one inflammatory cytokine to increase the opening of connexin-hemichannels (CxH);
b) collecting the cell culture supernatant;
c) culturing dendritic cells with the collected cell culture supernatant, or a fraction thereof or with a purified peptide from said cell culture supernatant, to get specific tumor antigen peptide repertoire loaded and/or activated dendritic cells; and
d) optionally purifying said specific tumor antigen peptide repertoire loaded and/or activated dendritic cells.

3. The method for obtaining an activated tumor antigen-specific CTL comprising the steps of:
a) exposing in suitable conditions a tumor cell culture to a Pattern Recognition Receptor (PRR) agonist and/or to one inflammatory cytokine to increase the opening of connexin-hemichannels (CxH);
b) collecting the cell culture supernatant; and
c) co-culturing dendritic cells and CTLs with the cell culture supernatant, or a fraction thereof or with a purified peptide from the cell culture supernatant, to get activated tumor antigen-specific CTLs.

4. The method according to claim 2 wherein dendritic cells are autologous or HLA-compatible or semi-compatible allogenic dendritic cells.

5. The method according to claim 1 wherein in step a) the tumor cell culture is incubated for at least 30 minutes with a Pattern Recognition Receptor (PRR) agonist and/or to one inflammatory cytokine to increase the opening of connexin-hemichannels (CxH).

6. The method according to claim 1, wherein the tumor cell culture is incubated at a temperature of 25-50° C. with a Pattern Recognition Receptor (PRR) agonist and/or to one inflammatory cytokine to increase the opening of connexin-hemichannels (CxH).

7. The method according to claim 1 wherein in step a) the tumor cell culture is incubated for 1 hour and half at 37° C. with a Pattern Recognition Receptor (PRR) agonist and/or to one inflammatory cytokine to increase the opening of connexin-hemichannels (CxH).

8. The method according to claim 1 wherein said cell culture supernatant is obtained by centrifugation of cells.

9. The method according to claim 8 wherein after centrifugation the supernatant is filtered.

10. The method according to claim 1 wherein the supernatant comprises a peptide comprising an amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

11. The method according to claim 10 wherein the supernatant comprises peptides comprising an amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

12. The method according to claim 1 wherein the inflammatory cytokine is gamma-IFN.

13. The method according to claim 1, wherein the tumor cell is an established tumor cell line, or a combination of tumor cell lines expressing a specific tumor antigen peptide repertoire or a tumor cell isolated by a tumor affected subject.

14. The method according to claim 1, wherein the tumor cell derives from solid or non-solid tumors, including melanoma, lung carcinoma, ovarian cancer, pancreatic cancer, glioma, glioblastoma, hepatocellular carcinoma, bladder cancer, stomach cancer, colorectal adenocarcinoma, prostate adenocarcinoma, sarcoma, osteosarcoma, leukemia and T cell-lymphoma and the said specific tumor antigen peptide repertoire is specific for said tumor.

15. The method according to claim 1, wherein the PRR agonists are Gram-negative, or Gram-positive bacteria or components thereof.

16. The method according to claim 15 wherein Gram negative bacteria components are LPS and/or flagellin or wherein Gram positive bacteria component is Lipoteichoic acid (LTA).

17. A supernatant or a fraction thereof obtainable by the method according to claim 1.

18. The supernatant or a fraction thereof according to claim 17 comprising peptides characterized through mass spectrometry analysis by at least one of the pics selected from the pics represented in FIGS. 3 and/or 12.

19. The supernatant or fraction thereof according to claim 17 comprising a peptide comprising the amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

20. The supernatant or fraction thereof according to claim 19 comprising peptides comprising an amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

21. An isolated peptide comprising an amino acid sequence selected from the group consisting of: SEQ ID NO:3, SEQ ID NO:2, SEQ ID NO: 4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO: 15, SEQ ID NO: 16, SEQ ID NO:17, SEQ ID NO: 18, SEQ ID NO: 19, SEQ ID NO: 20, SEQ ID NO: 21, SEQ ID NO: 22, SEQ IS NO: 23, SEQ ID NO: 24, SEQ ID NO: 25, SEQ ID NO: 26, SEQ ID NO: 27, SEQ ID NO: 28, SEQ ID NO: 29, SEQ ID NO: 30, SEQ ID NO: 31 and SEQ ID NO: 32 or orthologues, variants or fragments thereof.

22. An isolated nucleic acid encoding the peptide or orthologues, variants or fragments thereof according to claim 21.

23. An expression vector capable of expressing a nucleic acid according to claim 22.

24.-29. (canceled)

30. A specific tumor antigen peptide repertoire loaded and/or activated dendritic cell obtainable by the method according to claim 2.

31.-34. (canceled)

35. A tumor antigen-specific CTL obtainable by the method according to claim 3.

36.-42. (canceled)


SEQUENCE LISTING (32 sequences)

SEQ ID NO: 1 - protein, 8 aa, Artificial Sequence
  H-2Kb restricted OVA octapeptide
SIINFEKL

SEQ ID NO: 2 - protein, 14 aa, Mus musculus
PTDAQGSASGNHSV

SEQ ID NO: 3 - protein, 15 aa, Mus musculus
YDATYETKESKKEDL

SEQ ID NO: 4 - protein, 12 aa, Mus musculus
REQAGGDATENF

SEQ ID NO: 5 - protein, 10 aa, Mus musculus
EEHPGGEEVL

SEQ ID NO: 6 - protein, 19 aa, Mus musculus
AVDKKAAGAGKVTKSAQKA

SEQ ID NO: 7 - protein, 13 aa, Mus musculus
ARPREEVVQKEQE

SEQ ID NO: 8 - protein, 12 aa, Mus musculus
YDQTVSNDLEEH

SEQ ID NO: 9 - protein, 11 aa, Mus musculus
KEQIQKSTGAP

SEQ ID NO: 10 - protein, 13 aa, Mus musculus
PTPQDAGKPSGPG

SEQ ID NO: 11 - protein, 11 aa, Mus musculus
AKADGIVSKNF

SEQ ID NO: 12 - protein, 11 aa, Mus musculus
AKAPTKAAPKQ

SEQ ID NO: 13 - protein, 12 aa, Mus musculus
AKEAAEQDVEKK

SEQ ID NO: 14 - protein, 13 aa, Mus musculus
AKEAAEQDVEKKK

SEQ ID NO: 15 - protein, 9 aa, Mus musculus
DNEYGYSNR

SEQ ID NO: 16 - protein, 15 aa, Mus musculus
GQVINETSQHHDDLE

SEQ ID NO: 17 - protein, 11 aa, Mus musculus
HLDKAQQNNVE

SEQ ID NO: 18 - protein, 10 aa, Mus musculus
IGDSGVGKSN

SEQ ID NO: 19 - protein, 10 aa, Mus musculus
IPSDSTRRKG

SEQ ID NO: 20 - protein, 16 aa, Mus musculus
KKVAPAPAVVKKQEAK

SEQ ID NO: 21 - protein, 11 aa, Mus musculus
KNLQTVNVDEN

SEQ ID NO: 22 - protein, 10 aa, Mus musculus
LQDSGEVRED

SEQ ID NO: 23 - protein, 13 aa, Mus musculus
NKSTESLQANVQR

SEQ ID NO: 24 - protein, 16 aa, Mus musculus
PDPAKSAPAPKKGSKK

SEQ ID NO: 25 - protein, 15 aa, Mus musculus
PEPAKSAPAPKKGSK

SEQ ID NO: 26 - protein, 12 aa, Mus musculus
PVVQPSVVDRVA

SEQ ID NO: 27 - protein, 11 aa, Mus musculus
RDGQVINETSQ

SEQ ID NO: 28 - protein, 17 aa, Mus musculus
RLSSLRASTSKSESSQK

SEQ ID NO: 29 - protein, 21 aa, Mus musculus
RSAVPPGADKKAEAGAGSATE

SEQ ID NO: 30 - protein, 8 aa, Mus musculus
TEEEKNFK

SEQ ID NO: 31 - protein, 15 aa, Mus musculus
TVETRDGQVINETSQ

SEQ ID NO: 32 - protein, 16 aa, Mus musculus
TVGGDKNGGTRVVKLR